(19) United States
(12) Patent Application Publication
(10) Pub. No.: US 2025/0186668 A1
(43) Pub. Date: Jun. 12, 2025

(54) DEVICES AND METHODS FOR ASSESSING VASCULAR ACCESS

(71) Applicants: The United States Government as Represented by the Department of Veterans Affairs, Washington, DC (US); CASE WESTERN RESERVE UNIVERSITY, Cleveland, OH (US)

(72) Inventors: STEVE MAJERUS, University Heights, OH (US); BINIT PANDA, Cleveland, OH (US); STEPHANIE CHIN, Cleveland, OH (US)

(21) Appl. No.: 17/780,354

(22) PCT Filed: Nov. 25, 2020
(86) PCT No.: PCT/US2020/062183

§ 371 Date: May 26, 2022

Related U.S. Application Data
(60) Provisional application No. 62/941,204, filed on Nov. 27, 2019.

(51) Int. Cl. A61M 1/36

(52) U.S. Cl. CPC A61M 1/3656; A61M 2205/0294; A61M 2205/3375

(57) ABSTRACT

An apparatus can be used for detecting acoustic signals of a vascular system. The apparatus can comprise at least one acoustic sensor. Each acoustic sensor can comprise a piezoelectric layer defining a first side and a second side, and a first annular electrode disposed on the first side of the piezoelectric layer. The first annular electrode can define a hole therethrough. A second annular electrode can be disposed on the second side of the piezoelectric layer disposed against the second. A polymer engagement layer can be positioned against the first side of the piezoelectric layer and disposed at least partially within the hole of the first annular electrode. Methods of data processing for data collected via the apparatus are also disclosed herein.

### CROSS-REFERENCE TO RELATED APPLICATION

This application claims priority to and the benefit of U.S. Provisional Application No. 62/941,204, filed Nov. 27, 2019, the entirety of which is hereby incorporated by reference herein.

### FIELD

This disclosure generally relates to systems, apparatuses, and methods for assessing vascular access and, in particular, using acoustic data to assess vascular access.

### BACKGROUND

Hemodialysis is a life-sustaining treatment for individuals with end stage renal disease. During the treatment, arterial blood is filtered in an extracorporeal circuit and returned to the venous system, most often through an upper extremity vascular access. Widely used vascular accesses include arteriovenous fistulas and grafts or central venous catheters. Long-term dialysis efficacy and the lifetime cost of treatment is heavily dependent on maintaining the patency of the vascular access.

Vascular access dysfunction is the leading cause of hospitalization for patients on hemodialysis, accounting for 20-30% of hospital visits. The dominant cause of arteriovenous access dysfunction is stenosis (vascular narrowing) which increases the risk of thrombosis (vascular occlusion caused by clotting). These two have a combined incidence of 66-73% in arteriovenous fistulas (AVFs) and 85% in arteriovenous grafts (AVGs). Vascular access maintenance is thus a key objective of Kidney Disease Outcomes Quality Initiatives and dialysis care in general. Regular monitoring for vascular access dysfunction at the point of treatment can identify patients at risk of access thrombosis before full loss of access patency. This approach is enabled by the high frequency of treatment, commonly three times per week. Early detection enables the use of imaging to rule out false positives and treatment planning to avoid emergency interventions. Prominent monitoring strategies include physical exam and dialysis-based measures such as venous pump pressure, blood recirculation, and dialysate flow mismatch. These strategies have sensitivity of 75-82% (physical exam) and 35-48% (dialysis equipment measures) for detecting dysfunctional vascular accesses. Monthly access blood flow measurement is widely used, but there is a lack of agreement over actionable cutoffs, and studies have reported sensitivities of 24-88% depending on cutoff threshold. By contrast, duplex Doppler ultrasound scanning has 91% sensitivity of detection, but is not typically used unless other evidence of vascular access dysfunction is detected.

Monitoring programs in the dialysis center must be efficient and objective to reduce the labor burden and impact to clinical workflow. Physical exam is increasingly disused as it requires additional time per patient and is a subjective measure requiring skill and experience. One important aspect of physical exam is auscultation, in which a stethoscope is used to detect bruits (pathological blood flow sounds caused by vascular stenosis or other abnormalities). Mathematical analysis of bruits, i.e. phonoangiograms (PAGs), has enabled stenosis estimation from bruits spectral properties. However, previous work on PAGs has been limited by using stethoscopes to record bruits from one or two locations on a vascular access.

### SUMMARY

Described herein, in various aspects, is an apparatus for detecting acoustic signals of a vascular system. The apparatus can comprise at least one acoustic sensor. The at least one acoustic sensor can comprise a structure defining a hole therethrough. A piezoelectric polymer layer can have a first side and an opposing second side. The piezoelectric polymer layer extends across the hole that extends through the structure. A first electrode can be disposed on the first side of the polymer layer. A second electrode can be disposed on the second side of the polymer layer. A polymer engagement layer can be positioned against the first side of the polymer layer and can be disposed at least partially within the hole that extends through the structure.

A method can comprise: applying a bruit enhancing filter to data collected using an apparatus as disclosed herein to generate bruit enhanced filtered data. A wavelet transform can be applied to the bruit enhanced filtered data to provide wavelet data.

The method can further comprise generating an auditory spectral flux waveform (ASF) from the wavelet data and generating an auditory spectral centroid waveform (ASC) from the wavelet data.

The method can further comprise performing a systole/diastole segmentation on the auditory spectral flux waveform and the auditory spectral centroid waveform.

Performing the systole/diastole segmentation on the auditory spectral flux waveform and the auditory spectral centroid waveform can comprise calculating at least one of: a mean value of a systole segment of the ASC, a root mean square (RMS) of a systole segment of the ASF, a difference between the mean value of the systole segment of the ASC and a mean value of a diastole segment of the ASC, or a product of the mean of the systole segment of the ASC and the RMS of the systole segment of the ASF.

The method can further comprise: determining a first time of a crossing of a threshold of the ASF for data from a first sensor; determining a second time of a crossing of the threshold of the ASF for data from a second sensor that is distal to the first sensor with respect to a blood flow direction; and calculating a difference between the first time and the second time.

The method can further comprise determining a degree of stenosis based on the difference between the first time and the second time.

In various aspects, the method can include performing a regression (e.g., a Gaussian process regression) on the ASC, the ASF, and time data to determine a degree of stenosis (DOS). machine learning classifiers can be used to classify the DOS within at least one range (e.g., mild, moderate, and severe). The machine learning classifiers can comprise a support vector machine.

A system can comprise an apparatus as disclosed herein and a computing device. The computing device can comprise at least one processor and a memory in communication with the at least one processor, wherein the memory comprises instructions that, when executed by the at least one processor, perform a method as disclosed herein.

Additional advantages of the invention will be set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

These and other features of the preferred embodiments of the invention will become more apparent in the detailed description in which reference is made to the appended drawings wherein:

FIG. 1 illustrates a schematic of clinical system and a sensor array, in accordance with embodiments disclosed herein.

FIG. 2 illustrates a plurality of schematics of a sensor of the sensor array as in FIG. 1 at various stages of manufacture.

FIG. 3A is a top view of a sensor array in accordance with embodiments disclosed herein. FIG. 3B is a side perspective view of a portion of the sensor array as in FIG. 3A.

FIG. 3C is a side view of the sensor array as in FIG. 3A with the array in a bent configuration. FIG. 3D is an end view of the sensor array as in FIG. 3A. FIG. 3E is a side view of the sensor array as in FIG. 3A.

FIG. 4 is a schematic of the sensor array of FIG. 1 interacting with a test subject.

FIG. 5 is a schematic of a system comprising a sensor array in accordance with embodiments disclosed herein.

FIG. 6 is another schematic of a system comprising a sensor array in accordance with embodiments disclosed herein.

FIG. 7 is yet another schematic of a system comprising a sensor array in accordance with embodiments disclosed herein.

FIG. 8A is a schematic for a test apparatus for testing the sensor arrays of FIG. 1.

FIG. 8B illustrates a schematic for a test apparatus for measuring cross-talk between the sensors of the sensor array of FIG. 1.

FIGS. 9A and 9B illustrate, respectively, PAG and spectrogram recorded for a vascular access phantom with low (10%) degree of stenosis (DOS). FIGS. 9C and 9D illustrate, respectively, PAG and spectrogram recorded for a vascular access phantom with high (80%) DOS.

FIG. 10 illustrates a method for processing PAG signals and extracting relevant features.

FIGS. 11A and 11C illustrate, respectively, a PAG signal and its spectrogram before a bruit-enhancing filter. FIGS. 11B and 11D illustrate, respectively, a PAG signal and its spectrogram after the bruit-enhancing filter

FIG. 12A shows a plot of an unprocessed PAG signal. FIG. 12B illustrates the data of FIG. 12A after a bruit-enhancing filter. FIG. 12C illustrates a continuous wavelet transform (CWT) of the acoustic data. FIG. 12D illustrates Auditory Spectral Flux (ASF) approximating a spectral first derivative for use to segment the waveform into segments for segmented feature extraction. FIG. 12E illustrates an Auditory Spectral Centroid (ASC).

FIG. 13A is a test system for testing the sensor array as in FIG. 1. FIG. 13B is a schematic diagram of an array positioned on the test system as in FIG. 13A. FIG. 13C is an image of a phantom test apparatus. FIG. 13D is a plurality of views showing different cross sectional areas along the phantom test apparatus as in FIG. 13C.

FIG. 14A illustrates PAG spectra for human patients. FIG. 14B illustrates inter-quartile range for each frequency. FIG. 14C illustrates a comparison between the tuned vascular phantom power spectrum and the average human spectrum.

FIG. 15 illustrates a test rig including a phantom and an exemplary sensor array.

FIG. 16 illustrates A̅S̅C̅_̅{̅S̅}̅ ·ASF_{RMS,S} features for mild (DOS<40%), moderate (40%<DOS<60%), and severe (DOS<60%).

FIG. 17 illustrates A̅S̅C̅_̅{̅S̅}̅ differences between adjacent locations on the phantom.

FIG. 18 illustrates a receiver operating characteristic (ROC) curve of A̅S̅C̅_̅{̅S̅}̅ ·ASF_{RMS,S}.

FIG. 19 illustrates a test board in accordance with the characterization tests.

FIG. 20A-E illustrate frequency responses for sensors with different backing layers.

FIG. 21 illustrates signal comparisons between sensors having different sizes and backings at multiple frequencies.

FIG. 22 illustrates analytical signals obtained from PAGs can include Hilbert envelope, auditory spectral flux, and frequency domain linear prediction-modeled (FDLP-modeled) envelopes.

FIG. 23 illustrates time and wavelet scale representations of PAG recorded from (A) conventional stethoscope, and (B) the 2 mm sensor with silicone gel, both using FDLP systole pulse enhancement.

FIG. 24A-B illustrate steps in the fabrication of a vascular stenosis phantom using a suture band around silicone tubing and cast into a bio-mimicking silicone rubber mold.

FIGS. 25A and 25B illustrate, respectively, a phonoangiogram and a wavelet transform of typical dialysis patient and vascular phantom without stenosis.

FIG. 26 illustrates a plot showing ASC values at different flow types and recording locations for phantoms with DOS between 10-80%.

FIG. 27 is a plot showing the average ASC value across physiologic flow levels at all three recording locations for phantoms with DOS between 10-80%.

FIG. 28 is a plot showing mean ASC values for all flow types recorded at Location 2 for each phantom.

FIG. 29A is a plot showing time-domain blood sound. FIG. 29B is a plot showing CWT spectral domain collected from a phantom, as disclosed herein, using a sensor array, as disclosed herein. FIG. 29C is a plot showing extracted analytic signal Auditory Spectral Flux. FIG. 29D is a plot showing systolic start/end times calculated by thresholding.

FIG. 30A is a plot showing ASF onset detection through multiple threshold points. FIG. 12B is a plot showing performance of Td, having a saturation of 25% of ASF RMS value. FIG. 30C is a plot showing threshold values of 5%, 25%, and 50%.

FIG. 31 is a plot showing that ASF calculated at proximal and distal locations show an inversion in T_{d}.

FIG. 32 is a plot showing T_{d} over the range of flow rates and DOS grade.

FIG. 33 is a plot showing time differences for each DOS grade.

FIG. 34 is a plot showing mean velocity change for each DOS grade.

FIG. 35 is a schematic of a system comprising a computing device, in accordance with embodiments disclosed herein.

FIG. 36 is a schematic diagram illustrating signal processing in the analog domain to maximize signal-to-noise ratio and prevent aliasing in analog to digital conversion and digital signal processing after digitation to extract features for classification.

FIG. 37 Is a plot showing power spectral density recorded at different locations relative to a 75% stenosis show a site-specific signal bandwidth. In general sites after stenosis have wider signal bandwidths because of the local presence of turbulent blood flow.

FIG. 38 is a bar graph showing a 95% power bandwidth for 156 PAG recordings for DOS 10-90% were aggregated based on recording site. Recordings at sites 2 and 3, indicate wider bandwidth independent of degree of stenosis or flow rate. This forms the basis of the classifier methodology, as there is a distinct correlation between elevated power and frequency content in the presence of stenosis.

FIG. 39 illustrates a chart showing analysis of interquartile range for PAGs recorded with DOS 10-90% showed a required bandwidth of at least 1,600 Hz to accurately capture signal dynamics in the analog signal processing section. Including a safety factor, the interface amplifier was designed for 2.25 kHz bandwidth to limit noise.

FIG. 40 is a schematic diagram of a polyvinylidene-fluoride (PVDF) transducer modeled simply as a resistor (R_{S}) and capacitor (Cₛ) in parallel with output current I_{signal} based on measured impedance at 100 Hz.

FIG. 41 is an exemplary plot of an op-amp open loop transfer function and noise transfer functions vs. frequency. Ideally, the noise transfer function will be flatter until the op-amp gain begins to roll off (e.g. “B”).

FIG. 42 is a schematic diagram of an equivalent transducer and transimpedance amplifier circuit model for input referred noise calculations with parallel current source, l̅ₙ̅, functioning as input noise source.

FIG. 43 is a plot showing a spectrogram of artificially generated test waveform with 6 single-tone frequencies from 100-1500 Hz. The ASF curve (lower) shows a spike at every change of frequency, approximating the spectral first derivative.

FIG. 44 is a plot showing a spectrogram of artificially generated test waveform with 6 single-tone frequencies from 100-1500 Hz. The ASC curve describes the frequency of the sine wave at each time point

FIG. 45 is a time-domain bruit (A) and continuous wavelet transform spectral domain (B). The descriptive signals Auditory Spectral Centroid and Flux were extracted from CWT coefficients (C,D). The RMS value of the descriptive signals is one example of a scalar feature derived from the time-domain waveform.

FIG. 46 is a plot of auditory spectral centroid (ASC) varies with degree of stenosis but also between systolic and diastolic phases (A) and a plot of the auditory spectral flux (ASF) waveform that enables segmentation between pulsatile phases so that the RMS value of ASC (ASC_{RMS}) can be separately calculated.

FIG. 47 is a diagram illustrating how features can be derived from each recording site and combined into a featureset for classification.

FIG. 48 illustrates a plot of ASF calculated at proximal and distal locations showed an inversion in T_{d}. At moderate and severe DOS, T_{d} became negative, suggesting flow velocity increase.

FIG. 49 illustrates a diagram showing data processing. As features are extracted the dimensionality of the dataset is reduced to yield a final set of features. Since each site has features extracted from site-specific features and intra-site feature differences, a total featureset of F[S,M] is produced with S features over M sites.

FIG. 50 is an exemplary diagram illustrating stenosis localization using spatial features derived from feature differences between adjacent sites. In this example, the shift in A̅S̅C̅S̅ between sites is used to detect the presence of stenosis beneath a specific recording site.

FIG. 51 illustrates plots showing differences in ASC_{S} between sensor locations. As illustrated, difference in ASC_{S} between adjacent locations showed no significant variation for 0% DOS (p>0.05) (A). A large spectral shift at locations distal to stenosis (stenosis center at location 2) (B). Data plotted for phantoms with 30%<DOS<90%, p<0.001 for all locations. Analysis of Variance and Tukey's test were identified statistically-significant differences in ASC means at significance level α=0.05.

FIG. 52 illustrates a diagram illustrating how Scalar features can be derived from the time-domain ASC and ASF descriptive waveforms, including interaction features such as A̅S̅C̅·̅A̅S̅F̅. Temporo-spectral features such as systolic width can be derived, or compared to adjacent sites to compute spatial features such as t_{d} which describes the time shift at ASF onset in systole between time-synchronous recordings.

FIG. 53A illustrates a plot of ASC Systolic Mean vs Mean ASC-ASF. FIG. 53B illustrates a plot of ASC Onset Time vs Mean ASC-ASF. The quadratic SVM classified DOS as mild (<30%), moderate (30%<DOS<60%), and severe (DOS>60%) with 100% accuracy. This demonstrates the advantage of SVM as the included features are not fully separable linearly in the feature space (A,B).

FIG. 54A is an exponential Gaussian process regression estimated degree of stenosis for each in vitro vascular stenosis phantom. FIG. 54B is a plot showing the trained model estimated degree of stenosis with RMS error of 4.3%. FIG. 54C is a plot showing error range of [−11% 14%] and [−11% 3%] for all tested stenoses and for stenoses >50%, respectively.

FIG. 55 is an exemplary system for training artificial intelligence (AI), as disclosed herein.

FIG. 56 is an exemplary method for using AI to determine degree of stenosis.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a,” “an,” and “the” include plural referents unless the context clearly dictates otherwise. For example, use of the term “an acoustic sensor” can refer to one or more of such acoustic sensors, and so forth.

All technical and scientific terms used herein have the same meaning as commonly understood to one of ordinary skill in the art to which this invention belongs unless clearly indicated otherwise.

As used herein, the terms “optional” or “optionally” mean that the subsequently described event or circumstance may or may not occur, and that the description includes instances where said event or circumstance occurs and instances where it does not.

As used herein, the term “at least one of” is intended to be synonymous with “one or more of.” For example, “at least one of A, B and C” explicitly includes only A, only B, only C, and combinations of each.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedent “about,” it is contemplated that values within up to 15%, up to 10%, up to 5%, or up to 1% (above or below) of the particularly stated value can be included within the scope of those aspects. Similarly, use of the antecedent “generally” (e.g., “generally circular”) can indicate variances of up to 15%, up to 10%, up to 5%, or up to 1%.

The word “or” as used herein means any one member of a particular list and also includes any combination of members of that list.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

The following description supplies specific details in order to provide a thorough understanding. Nevertheless, the skilled artisan would understand that the apparatus, system, and associated methods of using the apparatus can be implemented and used without employing these specific details. Indeed, the apparatus, system, and associated methods can be placed into practice by modifying the illustrated apparatus, system, and associated methods and can be used in conjunction with any other apparatus and techniques conventionally used in the industry.

As further disclosed herein, to enable prospective monitoring at the point of care or in a patient's home, large-scale microphone arrays can be combined with data recording and digital signal processing circuitry for real-time quantification of vascular access stenosis. Phonoangiograms (PAGs) can implement an established element of physical exam, are easy to measure from the skin surface, and are specifically produced by vascular access structures.

A bruit spectral analysis can separate systole and diastole components of blood flow at access points. Compared to using stethoscopes, microphone arrays have a significant benefit for pinpointing vascular abnormalities. Further, thin-film contact microphones have greater sensitivity and bandwidth than conventional stethoscopes. In a prospective monitoring scenario, microphone arrays can be used to simultaneously record from many sites along a long and tortuous vascular access. Correlated signal processing between channels can be used to reject ambient noise and detect site-to-site differences in bruit spectra. Classifier algorithms or other quantification techniques can then be used to automatically detect at-risk patients for follow-up imaging.

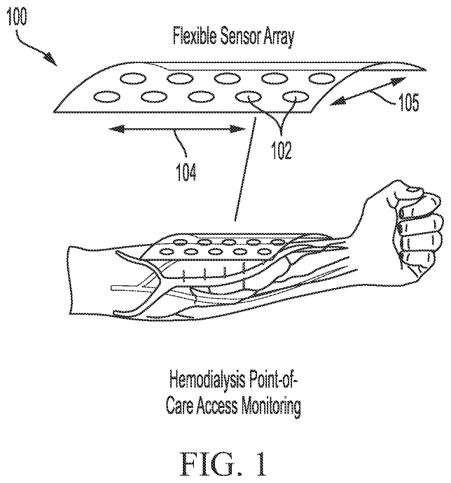

Disclosed herein, in various aspects and with reference to FIGS. 1-4, is an acoustic sensor array 100 comprising one or more sensors 102. Optionally, the sensors can be arranged along a first axis 104. In some optional aspects, the sensors 102 can be arranged in a non-linear arrangement to trace a pathway of a particular vein. In further embodiments, the sensors can be arranged in a two-dimensional grid having a first axis 104 and a second axis 105 that is perpendicular to the first axis 104. Each sensor 102 can comprise first electrode 106 and a second electrode 108. Optionally, each of the first electrode 106 and second electrode 108 can be an annular electrode defining a respective hole 110 therethrough. The holes 110 of the respective first and second electrodes 106, 108 of each sensor can be axially aligned. Optionally, the holes 110 can be between one and three millimeters in diameter, or about two millimeters in diameter.

A piezoelectric layer 112 can be disposed between the first electrode 106 and the second electrode 108 of each sensor 102 and can span across the holes 110 of the first electrode 106 and the second electrode 108. According to further optional aspects, another structure, such as, for example, a flexible printed circuit board, can define a hole therethrough, and the piezoelectric layer 112 can extend across said hole in the structure. Optionally, the piezoelectric layer 112 can comprise polymer. In further aspects, other piezoelectric transducer materials can be used, such as, for example, blended crystalline forms of nylon (e.g. nylon-11), piezoceramic materials such as lead zirconate titanate or bismuth sodium titanate, or charged electret films based on, e.g. amorphous fluoropolymer substrates.

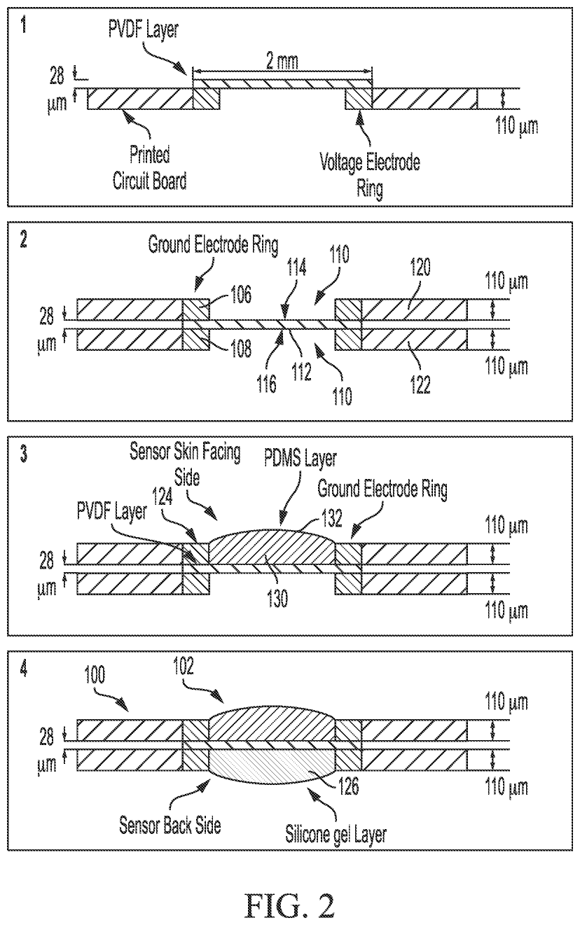

The piezoelectric layer 112 can have a first side 114 (shown as an upper side in FIG. 2) and an opposed second side 116 (shown as a lower side in FIG. 2). The piezoelectric layer 112 can optionally be between 1 and 100 microns thick, or about 28 micrometers thick. It is contemplated that the thickness can be selected to tune the acoustic performance of the sensor. The piezoelectric layer 112 can optionally comprise a silver ink metallized PVDF film. It is contemplated that the first electrode 106 and second electrode 108 can be in communication with respective areas of the silver ink for providing electrical readouts from the piezoelectric layer 112. PVDF material can have comparable recording properties compared to other piezoelectric crystals for frequency detection above 1 kHz and can be coated in flexible polymers such as polydimethylsiloxane (PDMS) to damp resonant modes or control frequency response in dynamic conditions. Differential layers of PDMS can be used for mechanical impedance matching. For example, optionally, a thin layer of soft PDMS can be used as the skin interface, and a layer of harder PDMS could be used to couple to the piezoelectric layer. Other elastomer materials beyond PDMS can be used to control the acoustic and mechanical performance, such as, for example, polyurethane, nitrile rubber, thermoplastics, etc.

The piezoelectric layer 112 can be polarized. Accordingly, fabrication methods can be selected to minimize heat exposure. For example, the piezoelectric layer 112 can be maintained at a temperature below 50° C. The piezoelectric layer 112 can be cut into a select size to span across the holes of the electrodes. The piezoelectric layer 112 can optionally be cut from a sheet with a laser cutter (Versa LASER, Models VLS2.30 and VLS 3.50). During laser cutting, compressed air (e.g., at 40 psi) can be jetted onto the substrate for cooling. Optionally, the laser cutting can be performed in two steps. For example, in one optional aspect, during manufacturing, an annular ring of metallization (for example, a ring of 0.5 mm) can first be removed from one sensor surface by raster-scanning (for example, at 14% power and 30% speed) to weaken the metal ink-PVDF bond without cutting through the piezoelectric layer 112. Weakened regions can be exfoliated by tape (e.g., Kapton tape) prior to cutting through the PVDF film to prevent shorting of the metal across the cut film. A final thickness cut (for example, a cut made at 17% power and 100% speed) can release each transducer element from the sheet. The settings (e.g., power and speed) can be dependent on the laser make/model and film thickness, silver thickness, etc.

The first electrode 106 of each sensor 102 can be arranged on a first printed circuit board (PCB) 120. Likewise, the second electrode 108 of each sensor 102 can be arranged on a second PCB 122. Each of the first PCB 120 and the second PCB 122 can optionally comprise a polyimide circuit board or other flexible circuit board. The first PCB 120 and second PCB 122 can each optionally comprise two-layer polyimide substrates that can be fabricated using copper (e.g., 0.5-oz copper) finished with nickel-gold plating. A polyimide overlay with solder contact openings can be applied to both boards for a total finished thickness that can optionally be about 110 μm. Each cut piezoelectric layer can be laminated between the first and second PCBs to electrically contact each side of the film separately. Optionally, the first and second electrodes 106, 108 of each sensor 102 can be attached to the piezoelectric layer 112 (e.g., PVDF film) using silver conductive epoxy adhesive (e.g., MG Chemicals, Model 8331). The first side 114 (skin-facing side) of the polymer layer 112 can be electrically grounded.

In further optional aspects, the second PCB 122 can be omitted. For example, in some optional embodiments, the first PCB 120 can define a hole therethrough, and the piezoelectric layer 112 can extend thereacross. The first electrode 106 can be in contact with the first side 114 of the piezoelectric layer 112. According to some aspects, the first electrode 106 can be an annular electrode as illustrated in FIG. 2 and further disclosed herein. The second electrode 108 can be in contact with the second side 116 of the piezoelectric layer 112. For example, the second electrode 108 can be deposited on, or otherwise coupled to, the backside of the piezoelectric layer 112, optionally, using conductive epoxy or other metallization. The second electrode 108 that is deposited on the backside of the piezoelectric layer 112 can optionally electrically couple to an electrode on the first PCB 120. The second electrode 108 can optionally have an annular shape.

It is further contemplated that only one of the first electrode 106 or the second electrode 108 is annular. In these aspects, the other of the first electrode 106 or the second electrode 108 on the opposing side of the piezoelectric layer 112 can be in contact with said opposing side of the piezoelectric layer 112. For example, the other of the first electrode 106 or the second electrode 108 can comprise a conductive epoxy that forms a bridge over a nonconductive region to connect to a pad on the first PCB 120 (optionally, with the second PCB omitted). It is contemplated that said configuration can be more easily and economically constructed. Accordingly, it is contemplated that an annulus or other structure (e.g., a PCB) defining a through-bore can support the diaphragm sensor (like a drum head), and the electrical contacts to the material can optionally be separate from the support structure. In various further aspects, only one of the electrodes is annular. In these aspects, the one annular electrode can comprise both conducive and non-conductive areas so that the electrode contact is not necessarily annular.

In various further optional aspects, the acoustic sensor array 100 can comprise a sheet of PVDF with the silver ink patterned onto it like a PCB (and otherwise omitting a PCB). The silver ink can be routed to an edge connector for providing electrical readouts. Accordingly, it is contemplated that, in various aspects, the acoustic sensor array 100 can comprise a plurality of electrodes distributed on a PCB or on a PVDF film can be altered as needed to improve the manufacturing/cost of the device.

The sensors 102 can be spaced so that outer edges of sequential sensors 102 are not physically connected, thereby minimizing crosstalk between the sensors 102. For example, the first PCB 120 and second PCB 122 can define spaces 140 between the sensors 102. That is, PCB material can be removed from between the sensors to form the spaces 140 between the sensors 102.

A contact layer 130 can cover the first side 114 of the piezoelectric layer 112. For example, optionally, the contact layer 120 can fill each hole 110 defined by the first electrode 106 (or the first PCB 120) so that a first side 132 of the contact layer extends above a skin-facing surface 124 of the first PCB 120. The contact layer 130 can extend sufficiently above the skin-facing surface 124 of the first PCB 120 so that the contact layer 130 can make contact with the skin to transmit acoustic waves from the skin to the piezoelectric layer 112. The contact layer 130 can optionally be about 1 mm in thickness. The contact layer 130 can optionally comprise PDMS (e.g., Ecoflex 00-10) with similar mechanical impedance, stiffness, and/or elastic modulus as muscle.

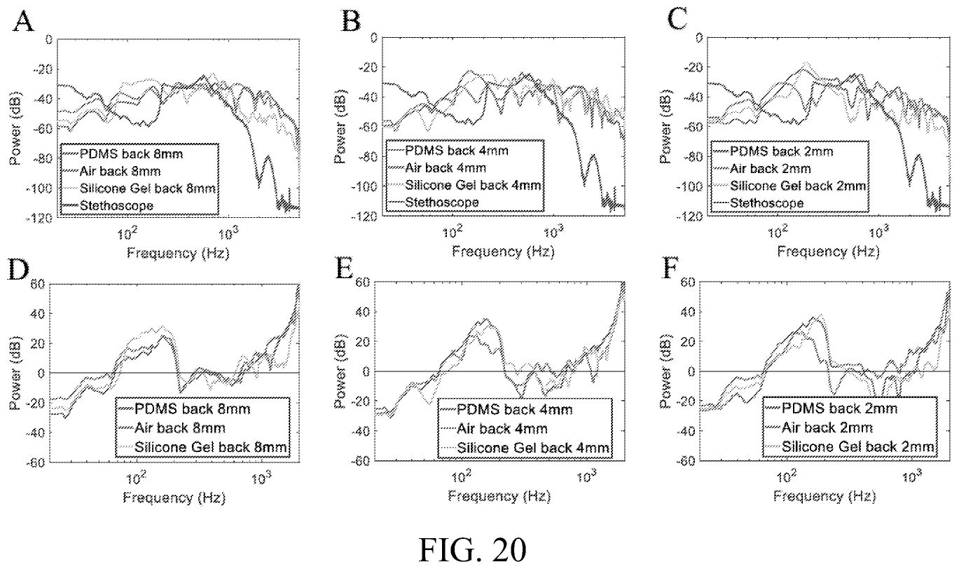

An outer layer 126 can cover the second side 116 of the piezoelectric layer 112. For example, outer layer 126 can comprise a silicone gel (e.g., Dow Corning SYLGARD 527 dielectric gel) and be sealed with polyimide tape. The outer layer 126 can optionally be about 80 to 140 μm thick, or more preferably, about 110 μm-thick. In various aspects, the thickness of the outer layer 126 can be selected to adjust acoustic performance of the sensor 102 (e.g., via damping). The outer layer 126 can enhance the acoustic properties of the sensor(s) 102 as shown in FIG. 20.

According to further aspects, an acoustic sensor array can comprise a plurality of integrated electronic microphones arranged in an array (e.g., soldered to a flexible circuit board). Such an array comprising integrated electronic microphones can be used for collecting acoustic signals, and the acoustic signals can be used for assessing vascular access, in accordance with embodiments disclosed herein.

### Systems and Methods

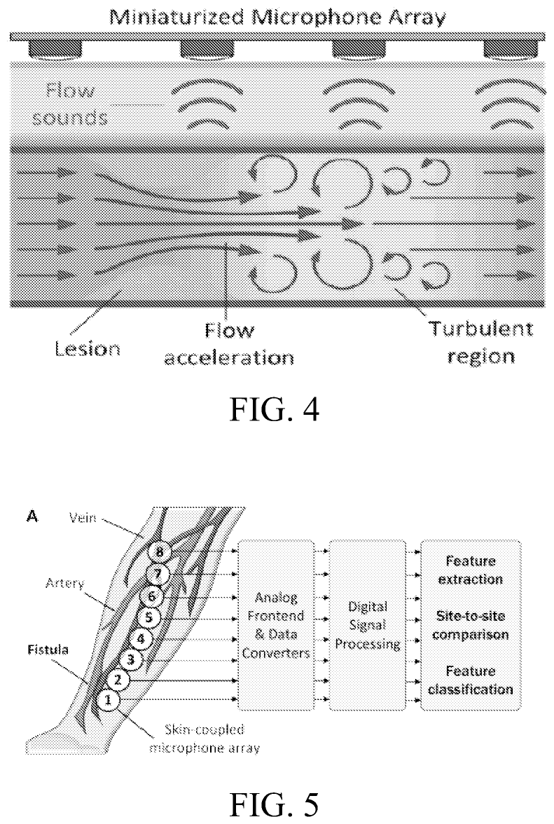

The sensor array 100 can be used to detect anomalies in vascular systems, such as, for example, a stenotic lesion. Referring also to FIG. 4, the stenotic lesion can cause turbulent blood flow at a distance of 1-3 times the vessel diameter distal to the stenosis. Turbulent vortices can create high-pitched blood flow sounds related to the degree of stenosis (DOS). The DOS can be defined as a ratio of the stenosed cross-sectional area of the blood vessel to the proximal, non-stenosed lumen diameter. According to some aspects, the stenosis can create turbulent flow when the DOS exceeds 50%.

Embodiments disclosed herein can detect acoustic frequencies in the range of 200-1000 Hz, which can be in excess of the neutral bandwidth of stethoscope diaphragms. DOS can be estimated by comparisons of proximal and distal recordings using multiple recording sites or a priori knowledge of the stenosis location. The location of stenosis can be estimated through recording at a plurality of sites along a vascular access and detecting regions of spectral variation caused by local turbulent flow. If a stenotic location is detected, DOS can be quantified by spectral analysis.

Due to the long length, tortuous, and variable anatomy of vascular accesses in patients, large-scale, flexible microphone arrays can be used to conform to the skin for accurate PAG recordings.

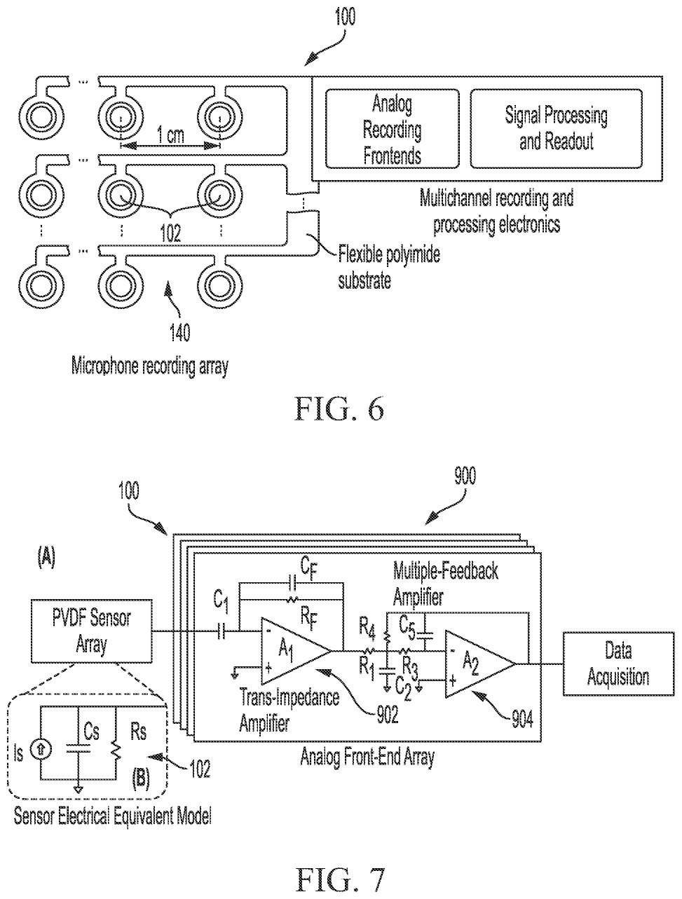

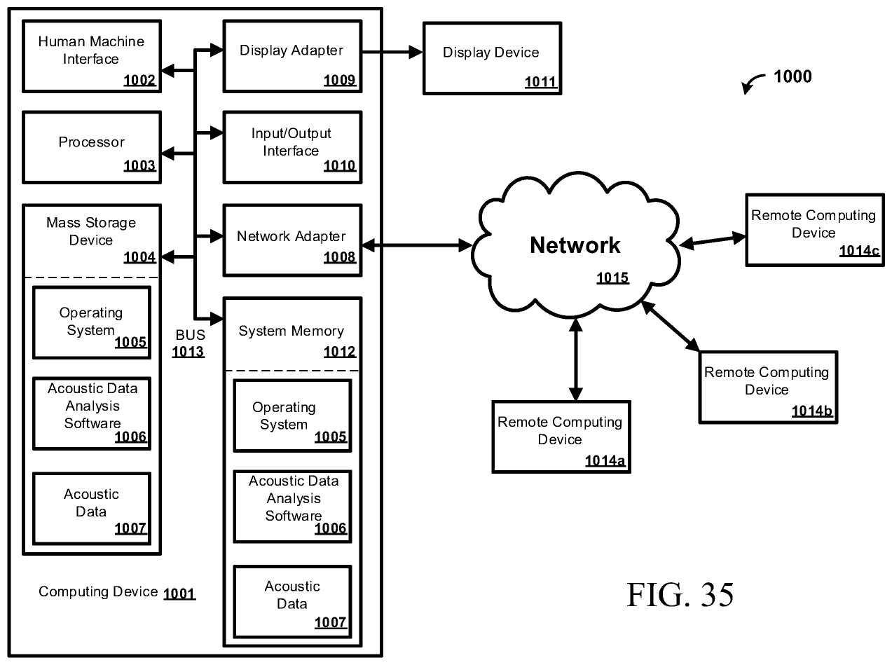

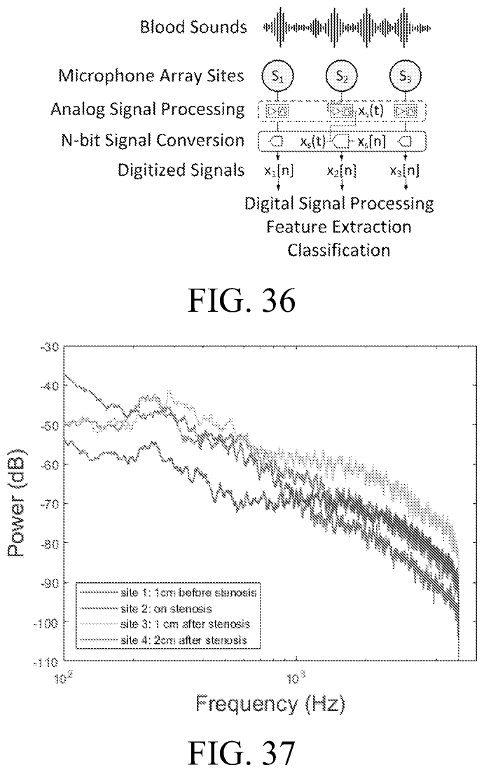

Referring to FIGS. 2 and 5-7, the acoustic sensor array 100 can be in communication with an analog front-end 902 and data converters 904 that can provide the signals from the acoustic sensor array 100 to a computing device 1001 (FIG. 35) that performs digital signal processing. Optionally, the analog front-end 902 (e.g., conditioning amplifiers, filters, etc.) and data converters 904 can be provided on one of the first or second PCBs 120,122. The same or a separate computing device 1001 can perform further data processing to analyze the data. For example, the computing device 1001 can extract features from the acoustic data, compare data between sensors, and classify features of the acoustic data.

### Computing Device

FIG. 35 shows a system 1000 including a computing device 1001 for use with the sensor array 100. In exemplary aspects, the computing device 1001 can be, for example, a personal computer, computing station (e.g., workstation), a portable computer, such as laptop computers, or a server.

The computing device 1001 may comprise one or more processors 1003, a system memory 1012, and a bus 1013 that couples various components of the computing device 1001 including the one or more processors 1003 to the system memory 1012. In the case of multiple processors 1003, the computing device 1001 may utilize parallel computing.

The bus 1013 may comprise one or more of several possible types of bus structures, such as a memory bus, memory controller, a peripheral bus, an accelerated graphics port, and a processor or local bus using any of a variety of bus architectures.

The computing device 1001 may operate on and/or comprise a variety of computer readable media (e.g., non-transitory). Computer readable media may be any available media that is accessible by the computing device 1001 and comprises, non-transitory, volatile and/or non-volatile media, removable and non-removable media. The system memory 1012 has computer readable media in the form of volatile memory, such as random access memory (RAM), and/or non-volatile memory, such as read only memory (ROM). The system memory 1012 may store data such as acoustic data 1007 and/or program modules such as operating system 1005 and acoustic data analysis software 1006 that are accessible to and/or are operated on by the one or more processors 1003.

The computing device 1001 may also comprise other removable/non-removable, volatile/non-volatile computer storage media. The mass storage device 1004 may provide non-volatile storage of computer code, computer readable instructions, data structures, program modules, and other data for the computing device 1001. The mass storage device 1004 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 1004. An operating system 1005 and the acoustic data analysis software 1006 may be stored on the mass storage device 1004. One or more of the operating system 1005 and the acoustic data analysis software 1006 (or some combination thereof) may comprise program modules and the acoustic data analysis software 1006. The acoustic data 1007 may also be stored on the mass storage device 1004. The acoustic data 1007 may be stored in any of one or more databases known in the art. The databases may be centralized or distributed across multiple locations within the network 1015.

A user may enter commands and information into the computing device 1001 via an input device (not shown). Such input devices comprise, but are not limited to, a keyboard, pointing device (e.g., a computer mouse, remote control), a microphone, a joystick, a scanner, tactile input devices such as gloves, and other body coverings, motion sensor, and the like. These and other input devices may be connected to the one or more processors 1003 via a human machine interface 1002 that is coupled to the bus 1013, but may be connected by other interface and bus structures, such as a parallel port, game port, an IEEE 1394 Port (also known as a Firewire port), a serial port, network adapter 1008, and/or a universal serial bus (USB).

A display device 1011 may also be connected to the bus 1013 via an interface, such as a display adapter 1009. It is contemplated that the computing device 1001 may have more than one display adapter 1009 and the computing device 1001 may have more than one display device 1011. A display device 1011 may be a monitor, an LCD (Liquid Crystal Display), light emitting diode (LED) display, television, smart lens, smart glass, and/or a projector. In addition to the display device 1011, other output peripheral devices may comprise components such as speakers (not shown) and a printer (not shown) which may be connected to the computing device 1001 via Input/Output Interface 1010. Any step and/or result of the methods may be output (or caused to be output) in any form to an output device. Such output may be any form of visual representation, including, but not limited to, textual, graphical, animation, audio, tactile, and the like. The display 1011 and computing device 1001 may be part of one device, or separate devices.

The computing device 1001 may operate in a networked environment using logical connections to one or more remote computing devices 1014a,b,c. A remote computing device 1014a,b,c may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the computing device 1001 and a remote computing device 1014a,b,c may be made via a network 1015, such as a local area network (LAN) and/or a general wide area network (WAN). Such network connections may be through a network adapter 1008. A network adapter 1008 may be implemented in both wired and wireless environments. Such networking environments are conventional and commonplace in dwellings, offices, enterprise-wide computer networks, intranets, and the Internet.

Application programs and other executable program components such as the operating system 1005 are shown herein as discrete blocks, although it is recognized that such programs and components may reside at various times in different storage components of the computing device 1001, and are executed by the one or more processors 1003 of the computing device 1001. An implementation of the acoustic data analysis software 1006 may be stored on or sent across some form of computer readable media. Any of the disclosed methods may be performed by processor-executable instructions embodied on computer readable media.

### Machine Learning

The computing device 1001 can comprise a machine learning module that is in communication with a memory module.

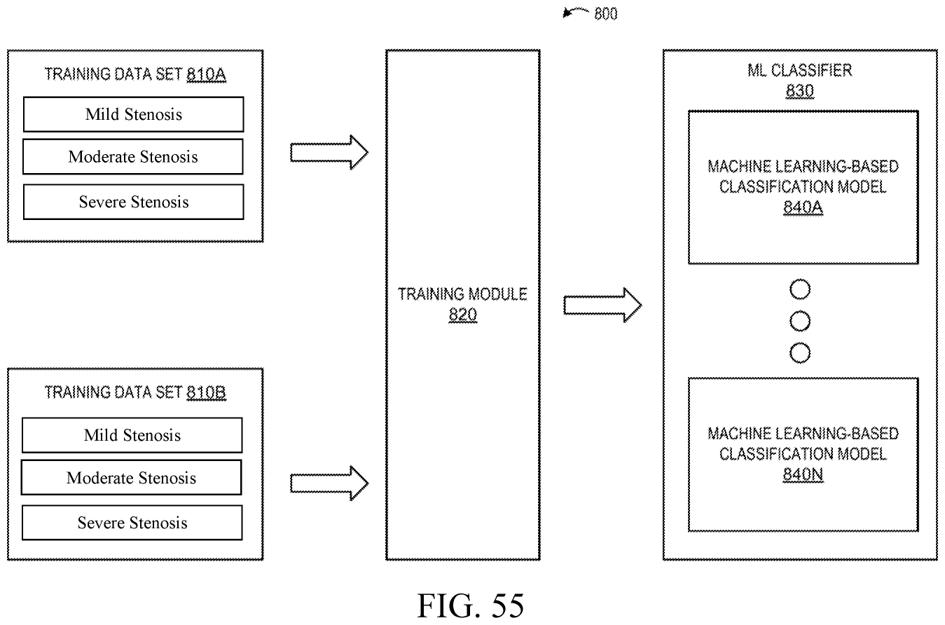

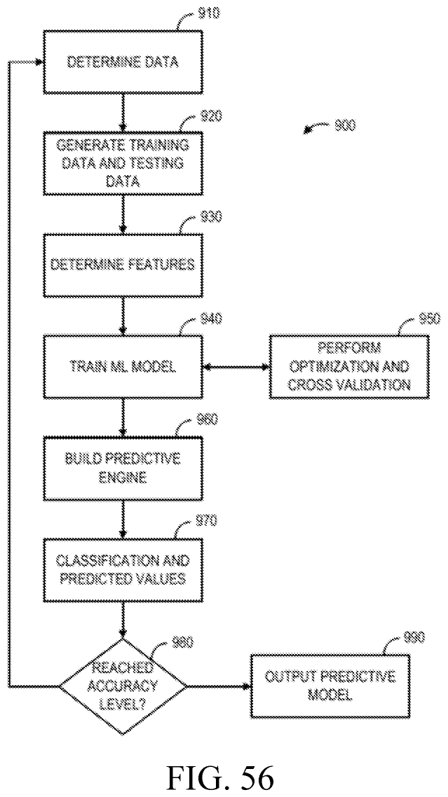

Turning now to FIG. 55, a system 800 is shown. The system 800 may be configured to use machine learning techniques to train, based on an analysis of one or more training data sets 810A-810B by a training module 820, at least one machine learning-based classifier 830 that is configured to classify acoustic signals as indicating a degree of stenosis range (e.g., mild, moderate, or severe). The training data set 810A (e.g., a first portion of the acoustic data from one or more sensors) can comprise acoustic features and an associated degree of stenosis that the acoustic features indicate. The training data set 810B (e.g., a second portion of the acoustic data from one or more sensors) can comprise acoustic features and an associated degree of stenosis that the acoustic features indicate. The labels may comprise “mild stenosis,” “moderate stenosis,” and “severe stenosis.”

The second portion of the acoustic data from one or more sensors can be randomly assigned to the training data set 810B or to a testing data set. In some implementations, the assignment of data to a training data set or a testing data set may not be completely random. In this case, one or more criteria may be used during the assignment, such as ensuring that similar numbers of acoustic data features with different associated degrees of stenosis are in each of the training and testing data sets. In general, any suitable method may be used to assign the data to the training or testing data sets, while ensuring that the distributions of sufficient quality and insufficient quality labels are somewhat similar in the training data set and the testing data set.

The training module 820 may train the machine learning-based classifier 830 by extracting a feature set from the first portion of the acoustic data from the one or more sensors in the training data set 810A according to one or more feature selection techniques. The training module 820 may further define the feature set obtained from the training data set 810A by applying one or more feature selection techniques to the second portion of the acoustic data from one or more sensors in the training data set 810B that includes statistically significant features of positive examples (e.g., features a particular attribute(s) of a given DOS) and statistically significant features of negative examples (e.g., features a particular attribute(s) outside of the given DOS).

The training module 820 may extract a feature set from the training data set 810A and/or the training data set 810B in a variety of ways. The training module 820 may perform feature extraction multiple times, each time using a different feature-extraction technique. In an embodiment, the feature sets generated using the different techniques may each be used to generate different machine learning-based classification models 840. For example, the feature set with the highest quality metrics may be selected for use in training. The training module 820 may use the feature set(s) to build one or more machine learning-based classification models 840A-840N that are configured to indicate whether or not new acoustic data features contain or do not contain features depicting a particular attribute(s) of the corresponding DOS.

The training data set 810A and/or the training data set 810B may be analyzed to determine any dependencies, associations, and/or correlations between extracted features and the sufficient quality/insufficient quality labels in the training data set 810A and/or the training data set 810B. The identified correlations may have the form of a list of features that are associated with labels for acoustic features depicting a particular attribute(s) of a corresponding acoustic data set and labels for acoustic features not depicting the particular attribute(s) of the corresponding DOS. The features may be considered as variables in the machine learning context. The term “feature,” as used herein, may refer to any characteristic of an item of data that may be used to determine whether the item of data falls within one or more specific categories. By way of example, the features described herein may comprise one or more acoustic feature attributes. The one or more acoustic feature attributes may include for example, a value related to (e.g., a mean of) at least a portion of the systole segment of the Auditory Spectral Centroid (ASC, further described herein) and a value of (e.g., an RSM of) at least a portion of the systole segment of the Auditory Spectral Flux (ASF, further described herein).

A feature selection technique may comprise one or more feature selection rules. The one or more feature selection rules may comprise an acoustic feature attribute and an acoustic feature attribute occurrence rule. The acoustic feature attribute occurrence rule may comprise determining which acoustic feature attributes in the training data set 810A occur over a threshold number of times and identifying those acoustic feature attributes that satisfy the threshold as candidate features. For example, any acoustic feature attributes that appear greater than or equal to 8 times in the training data set 810A may be considered as candidate features. Any acoustic feature attributes appearing less than 8 times may be excluded from consideration as a feature. Any threshold amount may be used as needed.

A single feature selection rule may be applied to select features or multiple feature selection rules may be applied to select features. The feature selection rules may be applied in a cascading fashion, with the feature selection rules being applied in a specific order and applied to the results of the previous rule. For example, the acoustic feature attribute occurrence rule may be applied to the training data set 810A to generate a first list of acoustic feature attributes. A final list of candidate features may be analyzed according to additional feature selection techniques to determine one or more candidate groups (e.g., groups of acoustic feature attributes). Any suitable computational technique may be used to identify the candidate feature groups using any feature selection technique such as filter, wrapper, and/or embedded methods. One or more candidate feature groups may be selected according to a filter method. Filter methods include, for example, Pearson's correlation, linear discriminant analysis, analysis of variance (ANOVA), chi-square, combinations thereof, and the like. The selection of features according to filter methods are independent of any machine learning algorithms. Instead, features may be selected on the basis of scores in various statistical tests for their correlation with the outcome variable (e.g., acoustic feature that depict or do not depict a particular attribute(s) of a corresponding acoustic data sample).

As another example, one or more candidate feature groups may be selected according to a wrapper method. A wrapper method may be configured to use a subset of features and train a machine learning model using the subset of features. Based on the inferences that drawn from a previous model, features may be added and/or deleted from the subset. Wrapper methods include, for example, forward feature selection, backward feature elimination, recursive feature elimination, combinations thereof, and the like. In an embodiment, forward feature selection may be used to identify one or more candidate feature groups. Forward feature selection is an iterative method that begins with no features in the machine learning model. In each iteration, the feature which best improves the model is added until an addition of a new feature does not improve the performance of the machine learning model. In an embodiment, backward elimination may be used to identify one or more candidate feature groups. Backward elimination is an iterative method that begins with all features in the machine learning model. In each iteration, the least significant feature is removed until no improvement is observed on removal of features. Recursive feature elimination may be used to identify one or more candidate feature groups. Recursive feature elimination is a greedy optimization algorithm which aims to find the best performing feature subset. Recursive feature elimination repeatedly creates models and keeps aside the best or the worst performing feature at each iteration. Recursive feature elimination constructs the next model with the features remaining until all the features are exhausted. Recursive feature elimination then ranks the features based on the order of their elimination.

As a further example, one or more candidate feature groups may be selected according to an embedded method. Embedded methods combine the qualities of filter and wrapper methods. Embedded methods include, for example, Least Absolute Shrinkage and Selection Operator (LASSO) and ridge regression which implement penalization functions to reduce overfitting. For example, LASSO regression performs 11 regularization which adds a penalty equivalent to absolute value of the magnitude of coefficients and ridge regression performs L2 regularization which adds a penalty equivalent to square of the magnitude of coefficients.

After the training module 820 has generated a feature set(s), the training module 820 may generate a machine learning-based classification model 840 based on the feature set(s). A machine learning-based classification model may refer to a complex mathematical model for data classification that is generated using machine-learning techniques. In one example, this machine learning-based classifier may include a map of support vectors that represent boundary features. By way of example, boundary features may be selected from, and/or represent the highest-ranked features in, a feature set.

The training module 820 may use the feature sets extracted from the training data set 810A and/or the training data set 810B to build a machine learning-based classification model 840A-840N for each classification category (e.g., each attribute of a corresponding acoustic data set). In some examples, the machine learning-based classification models 840A-840N may be combined into a single machine learning-based classification model 840. Similarly, the machine learning-based classifier 830 may represent a single classifier containing a single or a plurality of machine learning-based classification models 840 and/or multiple classifiers containing a single or a plurality of machine learning-based classification models 840.

The extracted features (e.g., one or more acoustic feature attributes) may be combined in a classification model trained using a machine learning approach such as discriminant analysis; decision tree; a nearest neighbor (NN) algorithm (e.g., k-NN models, replicator NN models, etc.); statistical algorithm (e.g., Bayesian networks, etc.); clustering algorithm (e.g., k-means, mean-shift, etc.); neural networks (e.g., reservoir networks, artificial neural networks, etc.); support vector machines (SVMs); logistic regression algorithms; linear regression algorithms; Markov models or chains; principal component analysis (PCA) (e.g., for linear models); multi-layer perceptron (MLP) ANNs (e.g., for non-linear models); replicating reservoir networks (e.g., for non-linear models, typically for time series); random forest classification; a combination thereof and/or the like. The resulting machine learning-based classifier 830 may comprise a decision rule or a mapping for each candidate acoustic feature attribute to assign an acoustic feature(s) to a class (e.g., depicting or not depicting a particular attribute(s) of a corresponding acoustic data set).

The candidate acoustic feature attributes and the machine learning-based classifier 830 may be used to predict a label (e.g., corresponding to a particular DOS) for the testing data set (e.g., in the second portion of the second acoustic data set). In one example, the prediction for each testing data set includes a confidence level that corresponds to a likelihood or a probability that the corresponding acoustic features depicts or does not depict a particular DOS. The confidence level may be a value between zero and one, and it may represent a likelihood that the corresponding acoustic feature(s) belongs to a particular class. In one example, when there are two statuses (e.g., depicting or not depicting a particular attribute(s) of a corresponding acoustic data sets), the confidence level may correspond to a value p, which refers to a likelihood that a particular acoustic feature belongs to the first status (e.g., depicting the particular attribute(s)). In this case, the value 1−p may refer to a likelihood that the particular acoustic feature belongs to the second status (e.g., not depicting the particular attribute(s)). In general, multiple confidence levels may be provided for each acoustic feature and for each candidate acoustic feature attribute when there are more than two statuses. A top performing candidate acoustic feature attribute may be determined by comparing the result obtained for each acoustic feature with the known sufficient quality/insufficient quality status for each corresponding acoustic data set in the testing data set (e.g., by comparing the result obtained for each acoustic feature with the labeled acoustic data of the second portion of the acoustic data from the one or more sensors). In general, the top performing candidate acoustic feature attribute for a particular attribute(s) of the corresponding acoustic data will have results that closely match the known depicting/not depicting statuses.

The top performing acoustic feature attribute may be used to predict the DOS based on acoustic features of a new acoustic data set. For example, a new acoustic data set may be determined/received. The new acoustic data set may be provided to the machine learning-based classifier 830 which may, based on the top performing acoustic feature attribute for the particular attribute(s) of the corresponding acoustic data, classify the acoustic features of the new acoustic data set as comprising or not comprising the particular attribute(s).

The application may provide an indication of one or more user edits made to any of the attributes indicated by a segmentation mask (or any created or deleted attributes) to the computing device 1001. For example, the user may edit any of the attributes indicated by the segmentation mask by dragging some of its points to desired positions via mouse movements in order to optimally delineate depictions of boundaries of the attribute(s). As another example, the user may draw or redraw parts of the segmentation mask via a mouse. Other input devices or methods of obtaining user commands may also be used. The one or more user edits may be used by the machine learning module to optimize the semantic segmentation model. For example, the training module 820 may extract one or more features from output data sets containing one or more user edits. The training module 820 may use the one or more features to retrain the machine learning-based classifier 830 and thereby continually improve results provided by the machine learning-based classifier 830.

Turning now to FIG. 9, a flowchart illustrating an example training method 900 is shown. The method 900 may be used for generating the machine learning-based classifier 830 using the training module 820. The training module 820 can implement supervised, unsupervised, and/or semi-supervised (e.g., reinforcement based) machine learning-based classification models 840. The method 900 illustrated in FIG. 9 is an example of a supervised learning method; variations of this example of training method are discussed below, however, other training methods can be analogously implemented to train unsupervised and/or semi-supervised machine learning models.

The training method 900 may determine (e.g., access, receive, retrieve, etc.) a first acoustic data set associated with a plurality of acoustic features (e.g., first acoustic data samples) and second acoustic data set associated with the plurality of acoustic features (e.g., second acoustic data samples) at step 910. The first acoustic data set and the second acoustic data set may each contain one or more acoustic result datasets associated with acoustic features, and each acoustic result datasets may be associated with a particular attribute. Each acoustic result data set may include a labeled list of acoustic results. The labels may comprise “mild stenosis,” “moderate stenosis, and severe stenosis.”

The training method 900 may generate, at step 920, a training data set and a testing data set. The training data set and the testing data set may be generated by randomly assigning labeled imaging results from the second acoustic data set to either the training data set or the testing data set. In some implementations, the assignment of labeled imaging results as training or test samples may not be completely random. In an embodiment, only the labeled acoustic results for a specific DOS type and/or class may be used to generate the training data set and the testing data set. In an embodiment, a majority of the labeled acoustic results for the specific DOS type and/or class may be used to generate the training data set. For example, 75% of the labeled acoustic results for the specific DOS type and/or class may be used to generate the training data set and 25% may be used to generate the testing data set.

The training method 900 may determine (e.g., extract, select, etc.), at step 930, one or more features that can be used by, for example, a classifier to differentiate among different classifications (e.g., “mild stenosis,” “moderate stenosis,” or “severe stenosis.”). The one or more features may comprise a set of acoustic data result attributes. In an embodiment, the training method 900 may determine a set features from the first acoustic data set. In another embodiment, the training method 900 may determine a set of features from the second acoustic data set. In a further embodiment, a set of features may be determined from labeled imaging results from a DOS type and/or class different than the DOS type and/or class associated with the labeled imaging results of the training data set and the testing data set. In other words, labeled imaging results from the different DOS type and/or class may be used for feature determination, rather than for training a machine learning model. The training data set may be used in conjunction with the labeled imaging results from the different DOS type and/or class to determine the one or more features. The labeled imaging results from the different DOS type and/or class may be used to determine an initial set of features, which may be further reduced using the training data set.

The training method 900 may train one or more machine learning models using the one or more features at step 940. In one embodiment, the machine learning models may be trained using supervised learning. In another embodiment, other machine learning techniques may be employed, including unsupervised learning and semi-supervised. The machine learning models trained at 940 may be selected based on different criteria depending on the problem to be solved and/or data available in the training data set. For example, machine learning classifiers can suffer from different degrees of bias. Accordingly, more than one machine learning model can be trained at 940, and then optimized, improved, and cross-validated at step 950.

The training method 900 may select one or more machine learning models to build a predictive model at 960 (e.g., a machine learning classifier). The predictive model may be evaluated using the testing data set. The predictive model may analyze the testing data set and generate classification values and/or predicted values at step 970. Classification and/or prediction values may be evaluated at step 980 to determine whether such values have achieved a desired accuracy level.

Performance of the predictive model described herein may be evaluated in a number of ways based on a number of true positives, false positives, true negatives, and/or false negatives classifications of acoustic features in acoustic data sets. For example, the false positives of the predictive model may refer to a number of times the predictive model incorrectly classified an acoustic feature(s) as depicting a particular attribute that in reality did not depict the particular attribute. Conversely, the false negatives of the machine learning model(s) may refer to a number of times the predictive model classified one or more acoustic features of acoustic data set as not depicting a particular attribute when, in fact, the one or more acoustic features do depicting the particular attribute. True negatives and true positives may refer to a number of times the predictive model correctly classified one or more acoustic features of an acoustic data set as having sufficient depicting a particular attribute or not depicting the particular attribute. Related to these measurements are the concepts of recall and precision. Generally, recall refers to a ratio of true positives to a sum of true positives and false negatives, which quantifies a sensitivity of the predictive model. Similarly, precision refers to a ratio of true positives a sum of true and false positives.

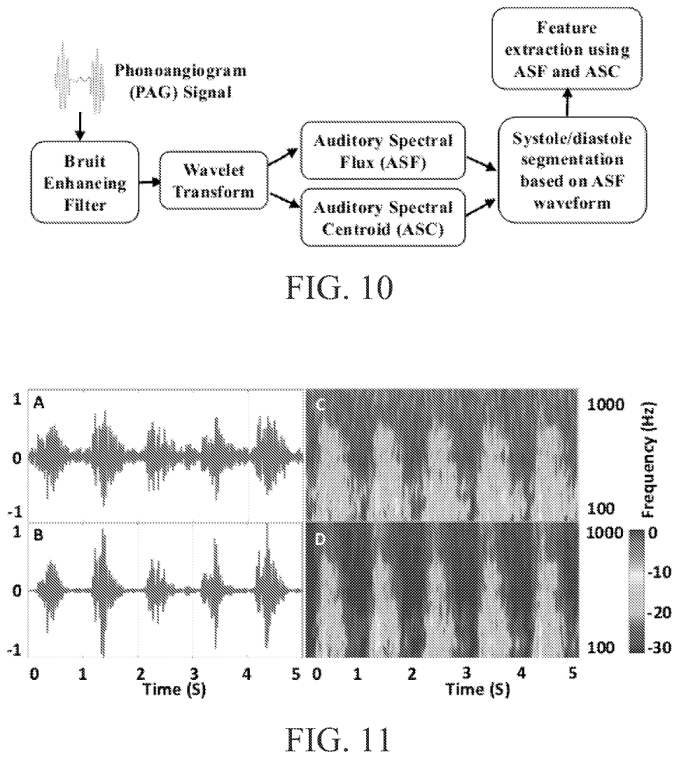

Further, the predictive model may be evaluated based on a level of mean error and a level of mean percentage error. For example, the predictive model may include an inference model. The inference model, as described herein, may be used to determine additional attributes associated with an acoustic data set, such as an amplitude or a frequency. FIGS. 10A and 10B show example evaluations of the inference model's predictions for amplitude and frequency values as compared to a trained expert.

Once a desired accuracy level of the predictive model is reached, the training phase ends and the predictive model may be output at step 990. When the desired accuracy level is not reached, however, then a subsequent iteration of the method 900 may be performed starting at step 910 with variations such as, for example, considering a larger collection of acoustic data.

### Exemplary Systems and Methods

It is contemplated that any content described in the following examples can be used to form an aspect of the disclosed systems and methods. Although described as separate examples, it is contemplated that particular parameters or steps of one example can be combined with parameters and steps of any other examples disclosed herein to produce additional aspects of the disclosed systems and methods. Thus, except as otherwise indicated, it is contemplated that steps or features of Example 1 can be combined with steps or features of Example 2. Similarly, except as otherwise indicated, it is contemplated that steps or features of Example 2 can be combined with steps and features of Example 1.

### Example 1

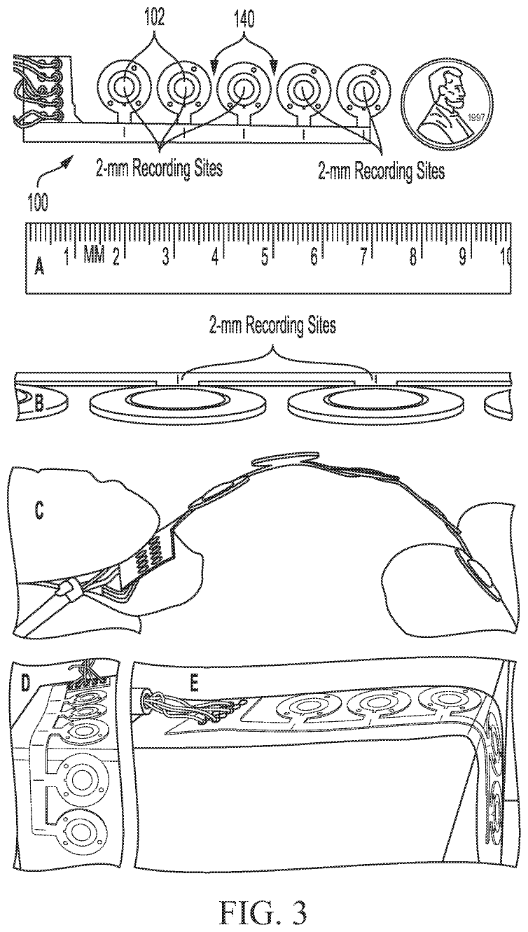

Referring to FIGS. 3A-E, an exemplary 5-channel sensor array can comprise a 2-mm PVDF sensor construction. The limited size of this array can be sufficient to determine sensor performance, including spatial crosstalk and stenosis localization on a vascular phantom (described below). Analog interface electronics can be assembled on a conventional rigid PCB and wired to the flexible microphone array. The array can be laid out in a 1×5 configuration with 1 cm spacing between microphone (sensor 102) centers (i.e., center-to-center). This 1 cm spacing can match the expected distance of turbulent blood flow occurring distal to stenosis. In further aspects, the center-to-center spacing can be between 0.2 cm and 2 cm. With this spacing, when arranged along a vein, at least one sensor 102 can be positioned to detect turbulent blood flow from stenosis within the vein. The polyimide PCBs can be laser-cut to remove extra material between microphone sites to reduce substrate coupling and crosstalk between channels. In this example, it is contemplated that assembled sensor arrays can bend up to 90 degrees with a bending radius of 0.6 cm.

The impedance of the PVDF microphones was measured for 10 assembled devices and averaged to develop a small-signal circuit model. An impedance analyzer (Hioki IM3570) was used to obtain the resistance and reactance from 5-5000 Hz and a parallel resistor/capacitor model was fitted. Average values over the tested frequencies were calculated to simplify the model (Table I). To model the equivalent output current for the PVDF film, a precision current amplifier (Stanford Research Systems SIM918) was used to convert current to voltage while recording PAGs from a calibrated vascular phantom. This phantom produced bruits with less than 2% spectral variance compared to those measured in humans. Typical sensitivity for the 2 mm PVDF microphone over a range of blood flow rates was 15 to 30 nAᵣₘₛ equivalent output current.

As shown in FIG. 7, the Analog Front End (AFE) model can comprise two stages. The first stage can be a trans-impedance amplifier (TIA) A₁ 910 to convert the microphone's output current to voltage with a 1.5-kHz low pass response to set the signal bandwidth, and the second amplifier A₂ 912 can be a multiple-feedback amplifier (MFA) and a low-pass filter with a cutoff at 2 kHz to limit pole splitting with the first stage dominant pole. Both stages can use the same CMOS op-amp (e.g., a Texas Instruments TLV9002) having design parameters are listed in Table I. PVDF sensor values were determined by an equivalent RC model measured at 100 Hz using an LCR Meter (Hioki IM3536). Sensor source current was measured with the sensor placed on a vascular phantom, to estimate the signal level when measuring PAGs from humans. Details of this phantom are disclosed herein. A precision current amplifier (Stanford Research Systems SIM918) was used to characterize the range of sensor current when measuring blood flow sounds from the phantom. These nominal sensor values were used to design the AFE transimpedance gain and bandwidth.

TABLE I
MODELED PARAMETERS OF THE PVDF SENSOR
AND DESIGN VALUES FOR ANALOG FRONT-END

| Parameter | Value | |
|---|---|---|
| Nominal sensor current (Is) | 15-30 | nA |
| Sensor modeled frequency | 100 | Hz |
| Modeled sensor capacitance (Cs) | 1 | nF |
| Modeled sensor resistance (Rs) | 12.4 | MΩ |
| TIA feedback impedance (RF, CF) | 1 MCI, 100 pF | |
| TIA trans-impedance | 1 | V/μA |
| TIA bandwidth | 1.5 | kHz |
| Simulated input-referred noise | 1.61 | pArms |
| MFA resistors (R1, R3, R4) | 5.23 kΩ, 7.5 kΩ, 182 kΩ | |
| MFA capacitors (C2, C5) | 22 nF, 200 pF | |
| MFA voltage gain | 33 | dB |
| MFA bandwidth | 2 | kHz |

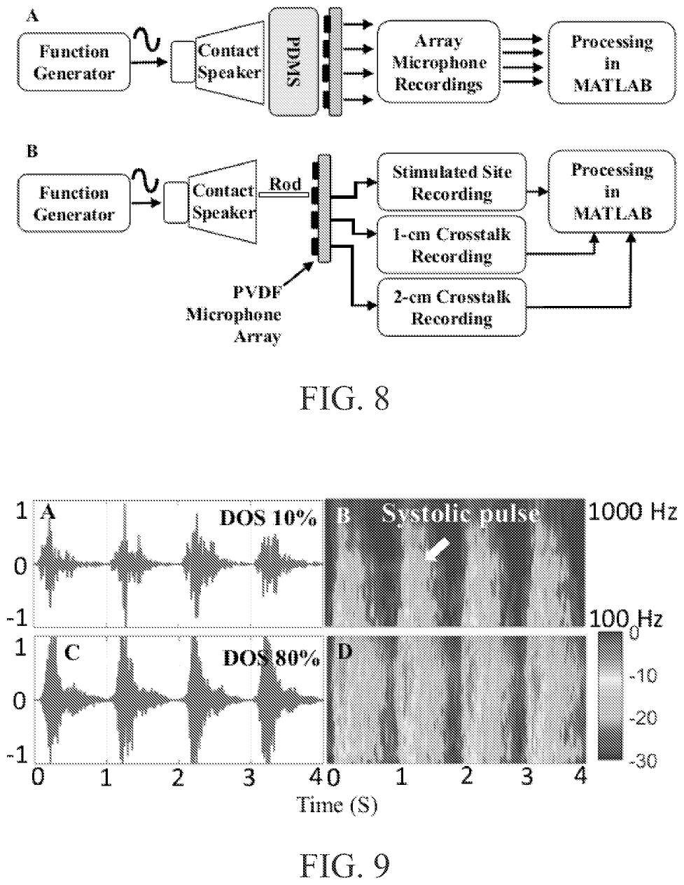

Referring to FIGS. 8A-B, the sensors were characterized for acoustic bandwidth and signal-to-noise ratio (SNR) using a bench setup. A function generator (Agilent 33522A) was used to drive a contact speaker with a variable frequency sinusoid signal from 1 to 10,000 Hz. To avoid harmonics due to overdriving, the driving voltage was limited to 50 mVpp. The SNR of the function generator source was measured at 100, 1000 and 1500 Hz using a dynamic signal analyzer (Stanford Research Systems Model SR 785) and the average SNR while driving the contact speaker was 80±2 dB. Low-frequency variation caused the function generator peak output voltage to vary by 1% throughout the test, which could have limited maximum SNR for devices under test. A 6 mm thick section of tissue-mimicking PDMS (Ecoflex 00-10) was placed on top of the speaker to mimic the impedance of human tissue (FIG. 8A). The frequency response of the setup was estimated by using a reference contact microphone (Tyco Electronics CM-01B) to find the inherent transfer function, thus accounting for the non-flat frequency characteristics of the contact speaker and PDMS sections. PVDF microphone recordings were processed through the inverse transfer function to obtain the frequency response. The SNR was also calculated with this setup using fixed tones at 100, 1,000, and 1,500 Hz. The averaged SNR values at each test frequency (Table II) confirmed that the proposed PVDF sensors have excellent SNR compared to similar recording devices.

TABLE II
MEASURED SNR OF ACOUSTIC SENSORS
AT 100, 1000 AND 1500 HZ

| Frequency | Stethoscope | SNR (mean ± SD) | PVDF |
|---|---|---|---|
| (Hz) | (n = 3) | Microphone (n = 9) | sensor (n = 9) |
| 100 | 18.7 | 15.5 ± 0.47 | 19.6 ± 4.2 |
| 1000 | 0.06 | 14.4 ± 0.2 | 26.9 ± 7.8 |
| 1500 | 0.018 | 15.0 ± 0.5 | 26.7 ± 8.6 |

Inter-channel Crosstalk Characterization: One aspect of importance with multichannel sensing is the effect of crosstalk between channels. For stenosis localization, low crosstalk is beneficial in detecting sudden changes in spectral content caused by turbulent blood flow. Crosstalk was measured on the bench using a similar setup as used for frequency response but with a different coupling method for the contact speaker (FIG. 8B). Instead of placing tissue-mimicking PDMS directly on the speaker, a 2 mm diameter, 10 cm long wooden rod was attached to the voice coil and positioned against the PDMS section to minimize acoustic spreading in the PDMS and approximate an acoustic point source. The point source was placed directly over one microphone in the array while recording from all other sites, and a 100-Hz test tone was applied. Crosstalk was measured as the power ratio of the excited microphone relative to other microphones in the array. The resulting power dropped approximately 41 dB at 1 cm and 53 dB at 2 cm, thus confirming low crosstalk (Table III).

TABLE III
MEASURED CROSSTALK BETWEEN
ADJACENT CHANNELS

| Distance from | Power (dB) | Crosstalk |
|---|---|---|
| Source (cm) | (mean ± SD) | value (dB) |
| 0 | 29.71 ± 1.12 | |
| 1 | −11.76 ± 4.96 | −41 |
| 2 | −23.81 ± 4.21 | −53 |

### Signal Processing

Conventional PAG signal processing techniques have used a variety of spectral approaches, including wavelet transform, short-time Fourier transform, and Burg autoregressive spectral estimates. However, all these methods have operated on aggregate PAG recordings, without time-domain segmentation. According to embodiments disclosed herein, a method for cardiac-cycle-based analysis can be performed in which systole and diastole segments are processed separately and spectral comparisons are performed differentially. This method has two distinct advantages. First, cardiac cycles can be treated as independent recordings, enabling averaging of values over multiple cycles to reduce spurious interference. Second, referring to FIG. 9, because fluid simulations estimate that turbulent flow occurs only during forward blood movement in systole, comparing the spectral gain in systole to diastole can reduce variability caused by changes in aggregate blood flow rate or ambient interference measured in both cycles. The spectral power increases in higher spectral bands when the DOS increases from 10% to 80%. Signal processing is used to quantify the properties of spectral change through feature calculation.

Referring to FIG. 10, the signal processing approach described herein can comprise multiple stages. First, raw PAG signals can be normalized to the same power level for each recording. This can reduce dependence on microphone sensitivity, AFE channel gain, and local pressure on each microphone during recording. This normalization does not affect spectral-based signal processing and feature extraction. PAGs can then be pre-processed using an autoregressive bruit enhancing filter. Next, auditory analytical waveforms can be calculated over entire recordings. Finally, waveforms are separated into systole and diastole segments, and features can be extracted. After the features are calculated, stenosis severity and location can be determined by classification methods.

### A. Autoregressive Bruit-Enhancing Filter

The bruit-enhancing filter (BEF) is based on sub-band frequency domain linear prediction (SB-FDLP) and enhances frequency components nonlinearly based on prominence. Effectively, the BEF enhances the systole portion of recordings because it is in this portion that most high frequency power occurs. A secondary effect of the BEF is to reduce skin scratch and pop noise artifacts obtained due to movement of the recording transducer over the skin. The development of the BEF was based on recordings from hemodialysis patients and described previously. Briefly, SB-FDLP envelopes are calculated from the discrete-cosine transform (DCT) values of the PAG using linear predictive coding (LPC) of the DCT coefficients. The DCT can approximate the envelope of the Discrete Fourier Transform. This can indicate that the spectrogram of the DCT (treating the DCT as a time sequence) can mirror the time-domain spectrogram around the time/frequency axes. When applied in the time domain, LPC estimates the frequency response as an autoregressive model. FDLP applies LPC to frequency-domain DCT coefficients to form an autoregressive model for the time-domain envelope. This implementation uses LPC to model the spectral envelope using a P-th order, all-pole finite impulse response (FIR) filter defined as

$$\hat{x} [ n ] = {\sum }_{k = 1}^{P} {a}_{x} [ n - k ] \to P ( z ) = {\sum }_{k = 1}^{P} {a}_{k} {z}^{- k}$$

where P is the order of the filter polynomial and P(z) is its z-transform. LPC uses least-squares iterative fitting to determine the coefficients aₖ of the filter P(z) such that the error in determining the next value of a series \hat{x}[n] is minimized. The calculated filter can be an autoregressive model with significantly lower variance than the Hilbert envelope, aiding in noise rejection. SB-FDLP simply applies the LPC to sub-bands of the DCT coefficients, i.e. P(z) in each individual band is represented as Hₘ(z) where m is for the m-th sub-band. Thus, the impulse response of Hₘ(z), denoted by Hₘ[n], can predict the time-domain envelope produced by frequencies of x[n] within the m-th sub-band. Because the poles of Hₘ(z) are fitted by order of prominence, only the most prominent time-domain occurrences of each sub-band contribution are approximated by the sub-band envelopes.

The N sub-band envelopes Hₘ[n] can be finally combined to produce a bruit-enhanced envelope E_{BEF}[n] using sub-band weights Wₘ, as follows:

$${E}_{BEF} [ N ] = {\sum }_{m = 1}^{N} {W}_{m} {H}_{m} [ n ] .$$

The sub-band weights, chosen empirically based on over 3,000 PAG recordings, are shown in Table IV. After E_{BEF}[n] is constructed, the original signal x[n] is multiplied by E_{BEF}[n] to produce the bruit-enhanced output signal x_{PAG}[n]=x[n]·E_{BEF}[n].

TABLE IV
SELECTED BANDS FOR FREQUENCY-DOMAIN MODELING

| | |
|---|---|
| Sub-band width | Sub-band weight |

| | | | |
|---|---|---|---|
| Band 0 | 25-225 | Hz | 5% |
| Band 1 | 350-700 | Hz | 50% |
| Band 2 | 650-1000 | Hz | 30% |
| Band 3 | 950-1200 | Hz | 15% |

FIG. 11A illustrates a PAG signal before the bruit-enhancing filter; FIG. 11B illustrates a spectrogram before the bruit-enhancing filter; FIG. 11C illustrates the PAG signal after the bruit-enhancing filter; FIG. 11D illustrates the spectrogram after the bruit-enhancing filter.

### B. PAG Auditory Feature Calculation Analysis

After the enhanced PAG is produced, a continuous wavelet transform (CWT) is computed to describe the spectral variance over time. The wavelet transform over k scales W[k, n] can be computed as follows:

$$W [ k , n ] = {x}_{PAG} [ n ] * ψ [ n / k ] ,$$

where ψ[n/k] is the analyzing wavelet at scale k. The complex Morlet wavelet can be used because it has a good mapping from scale to frequency.

Next, two fundamental n-point waveforms can be calculated from the set of CWT coefficients: Auditory Spectral Flux (ASF) and Auditory Spectral Centroid (ASC). Both ASF and ASC are spectral analytical signals from which auditory features can be extracted.

ASF describes the rate at which the magnitude of the auditory spectrum changes, and approximates a spectral first-order difference or first derivative. It can be calculated as the variation between two adjacent time frames:

$$A S F [ n ] = \frac{1}{K} \sqrt{{\sum }_{k = 1}^{K} {( \vert W [ k , n ] \vert - \vert W [ k , n - 1 ] \vert )}^{2}}$$

where W[k, n] is the CWT of the PAG obtained over a total of K scales.

ASC can describe the spectral “center of gravity” at each point in time, and is commonly used to estimate the average pitch of audio recordings. For Gaussian-distributed white noise (same spectral power at all frequencies), ASC can be centered at a pseudofrequency F[K/2]. Higher values of the centroid can correspond to “brighter” textures with more high frequency content. It can be defined as

$$ASF [ n ] = \frac{{\sum }_{k = 1}^{K} ( \vert W [ k , n ] \vert \cdot {f}_{c} [ k ] )}{{\sum }_{k = 1}^{K} ( \vert W [ k , n ] \vert )}$$

where f_{c}[k] is the center frequency for the k-th CWT scale.

### C. Cardiac Cycle Segmentation of PAG Analytical Signals

During pulsatile blood flow, the high velocity flow in systole can produce significant turbulence, causing the characteristic pitch of the bruit. Therefore, the systolic portion of the bruit can contain spectral content related to turbulent flow and can be analyzed separately from diastole to improve sensitivity. A segmentation technique can be implemented to segment the pulse into systole and diastole periods. Segmentation can rely on analyzing the time-domain ASF waveform to identify these epochs. First, 50% of the RMS value of the ASF waveform can be calculated as a threshold to identify the start and end of each cardiac cycle.

An initial identification of systole and diastole phases can be performed by locating alternating threshold crossings (below threshold to above threshold for start of systole, and the opposite for end of systole). Then selected systolic segments can be filtered through two stages. This filtering can reduce threshold double-crossings or crossings caused by transient recording artifacts. In the first stage, all detected segments can be considered as candidates, while, in the second stage, valid segments can meet two conditions: maximum systolic segment length less than 1 second and greater than 40% of the longest systolic segment from stage 1. This can remove spurious segments caused by recording noise peaks.

### D. Segment-Based Spectral Feature Extraction

Thirteen acoustic features can be calculated from PAG recordings. ANOVA and principal component analysis (PCA) were used to determine that ASC mean value (A̅S̅C̅_̅{̅S̅}̅ ) and ASF RMS value (ASF_{RMS}) can be the two most sensitive for detecting the DOS. The performance of these features can be further enhanced by calculating them separately for systole and diastole cardiac cycle segments. The segmented features can then be used independently and through linear combinations to provide augmented features with improved sensitivity. Statistical testing can again be used to determine the 4 segment features most strongly variable with DOS, namely, systole ASC mean (A̅S̅C̅_̅{̅S̅}̅ ), systole ASF RMS (ASF_{RMS,S}), difference between systole ASC and diastole ASC means (A̅S̅C̅_̅{̅S̅}̅ −A̅S̅C̅_̅{̅D̅}̅), and multiplication of systole ASC mean and systole ASF RMS (A̅S̅C̅_̅{̅S̅}̅ ·ASF_{RMS,S}).

These parameters are defined as follows. First,

$$\overline{{ASC}_{S}} = \frac{1}{D - S} , {\sum }_{n = S}^{D} ASC [ n ]$$

where D and S are the start times of a diastole and systole segment respectively. Second,

$$\overline{{ASC}_{S}} - \overline{{ASC}_{D}} = \frac{1}{D - S} {\sum }_{n = {S}_{i}}^{{D}_{i}} - \frac{1}{{S}_{i + 1} - D} {\sum }_{n = {D}_{i}}^{{S}_{i - 1}} ASC [ n ]$$

where Dᵢ and Sᵢ are the start times of the i^{th} diastole and systole segments, respectively. Finally, the RMS ASF value for each systole segment is calculated as

$${ASF}_{RMS , S} = \frac{1}{D - S} {\sum }_{n = S}^{D} {( \vert ASF [ n ] \vert )}^{2}$$

E. Example of PAG Feature Extraction from a Human Bruit Recording

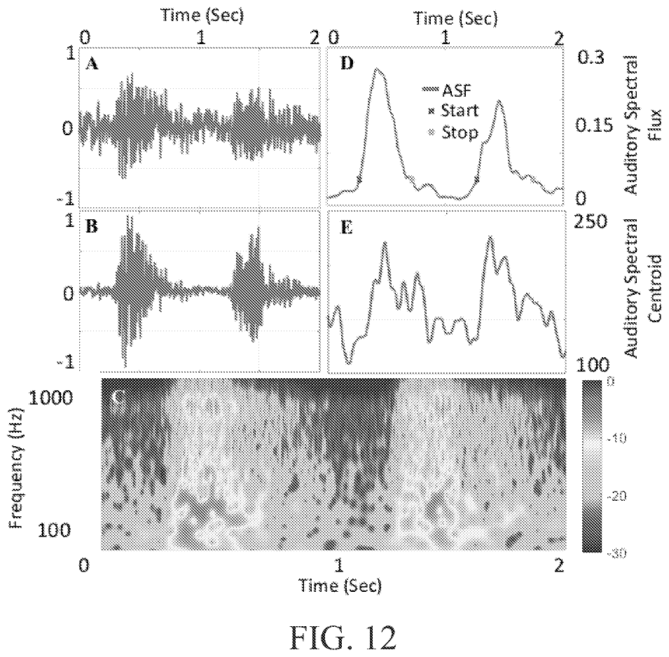

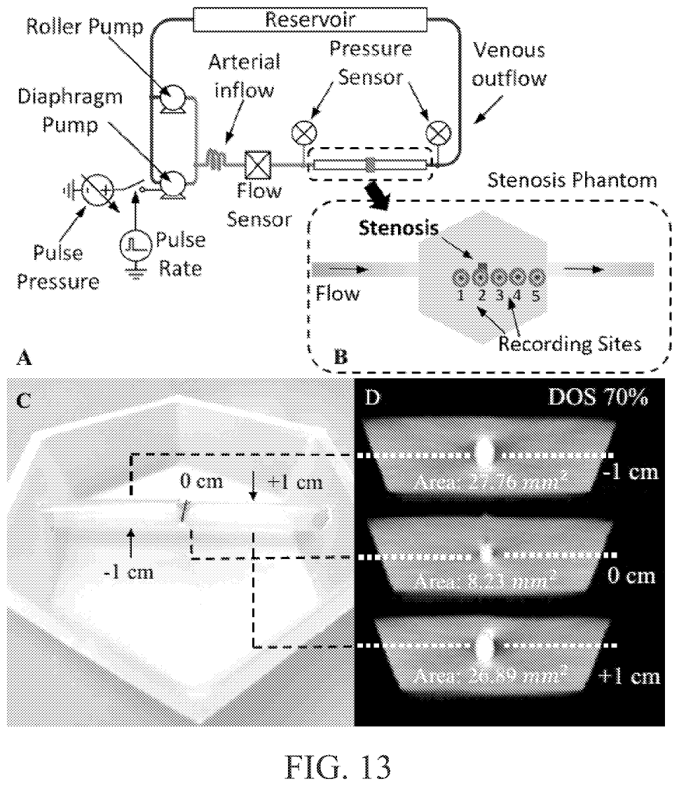

Human bruit recordings were recorded from 24 hemodialysis patients over 18 months and can be processed using the same technique to demonstrate the signal processing steps (FIG. 12B). First the raw signal is enhanced using bruit-enhancing filter. CWT of the enhanced PAG is next computed (FIG. 12C) and the coefficients are used to calculate ASF and ASC (FIGS. 12D and 12E, respectively). ASF can then be used to segment the waveforms into systole and diastole segments (FIG. 12D). Then (A̅S̅C̅_̅{̅S̅}̅ ), (ASF_{RMS,S}), (A̅S̅C̅_̅{̅S̅}̅ −A̅S̅C̅_̅{̅D̅}̅), (A̅S̅C̅_̅{̅S̅}̅ ·ASF_{RMS,S}) can be calculated as discussed herein.

### Stenosis Localization and Quantification Performance on Vascular Access Phantom

To simulate how the sensor can perform on a real patient (but in a controlled environment), recordings can be made on a vascular access phantom. One embodiment of a phantom can comprise a six mm silicone tube embedded in PDMS rubber (Ecoflex 00-10) at a depth of 6 mm. Stenosis can be simulated in the center of the phantom with a silk band tied around the tube to produce an abrupt narrowing. The DOS can be controlled by tying the band around metal rods with fixed diameters (FIG. 13C). DOS can be confirmed by calculating the percentage of lumen diameter reduction from CT scans of phantoms (FIG. 13D).

The phantom can be connected to a pulsatile flow pumping system (Cole Parmer MasterFlex L/S, Shurflo 4008) to simulate human hemodynamic flow from 700-1200 ml/min. Pulsatile pressures and aggregate flow rate were measured with a pressure sensor (PendoTech N-038 PressureMAT) and flow sensor (Omega FMG91-PVDF) respectively (FIG. 13A).

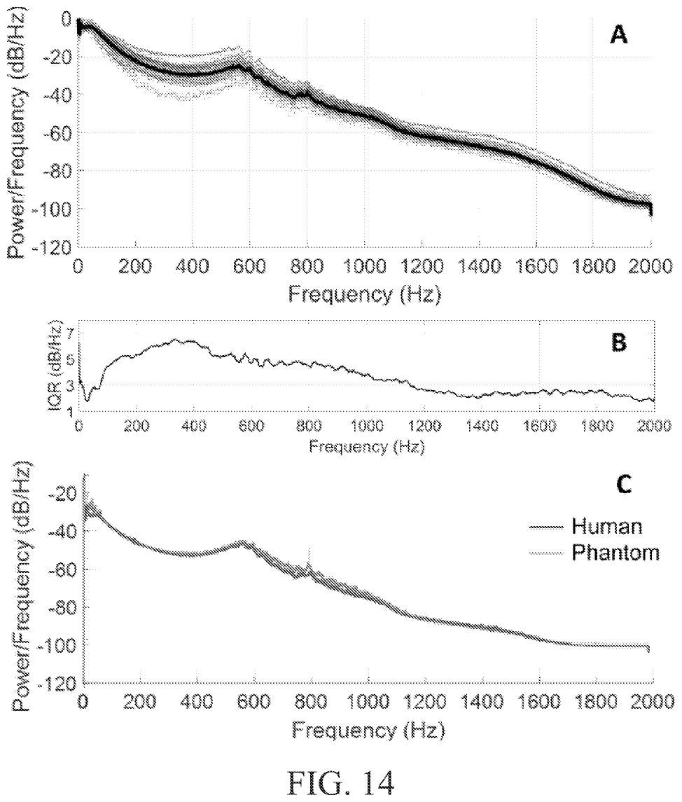

To validate the phantom auditory signals, recordings were made using a digital stethoscope (Littman 3200) and compared to recordings from humans. An aggregate power spectrum was produced from 3,441 unique ten-second recordings obtained from 24 hemodialysis patients over 18 months, as shown in FIG. 14. Despite large anatomical differences in this patient group, the interquartile range of all measurements from the mean spectrum was within 3-5 dB. The vascular phantoms were therefore tuned to produce a power spectrum to match the aggregate human power spectrum with the expectation that this would capture the range of human spectral power within 5 dB. Spectral comparisons between human and phantom data were used to reduce time-domain variability, for example due to heart rate differences. Human and phantom spectra followed similar spectral trends and showed similar dynamic range in time-domain analysis. Quantitatively, the normalized RMS error of the aggregate power spectrum was calculated over the aggregate human PAG spectra range. The vascular phantoms matched the aggregate human PAG spectra with a 1.84% normalized RMS error, scaled to the total power spectrum range. This comparison suggested that the vascular stenosis phantoms adequately replicated PAGs as measured from humans, but with control over vascular access flow rate and stenosis severity.

A. Stenosis Severity Detection from Recorded PAGs at Variable Flow Rates

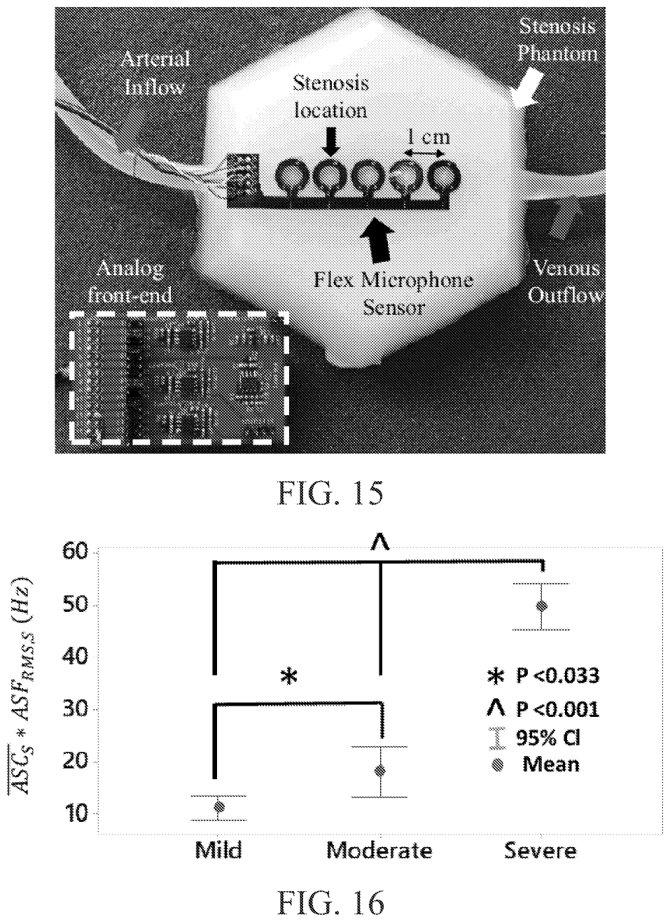

PAGs were investigated from 10 phantoms, shown in FIG. 15, with DOS from 10-85% to simulate a varying patient population. A set of ten second recordings were made with the flexible sensor array (discussed earlier) at six flow rates (700-1200 mL/min) to represent nominal physiological flow rates in functional vascular accesses. PAGs were recorded at four locations simultaneously spaced 1 cm apart, with one being proximal, one on the stenosis and two others distal to stenosis with Labview software and National Instruments data acquisition hardware at a sampling rate of 10 kHz. Five gram weights were placed on the back of each sensor to produce 50 mN of contact force. This light force reduced vibration artifacts and improved recording sensitivity. After recording, signals were transferred to MATLAB for signal processing and calculation of ASF and ASC waveforms. Signals were segmented into systole and diastole phases and features calculated separately. Because the ASF had a significant pulsatile component, RMS ASF values were used; otherwise, mean ASC values were calculated in each cardiac segment.

Features can be extracted for each PAG. These features can be based on ASC and ASF values that are analyzed with two primary outcomes: localization of the stenosis, and differentiation of the DOS. Balanced Analysis of Variance (ANOVA) can be conducted to detect differences in PAGs at different locations and at different DOS over a range of flows. ANOVA can be used to test the differences between means for statistical significance. In one test, the total number of recordings were 50 per location including all phantoms and flow rates. After segmentation there were approximately 350 systole and diastole segments for each phantom-location combination in this analysis.

A̅S̅C̅_̅{̅S̅}̅ ·ASF_{RMS,S} performed well in differentiating degree of stenosis in 3 grades: mild (DOS<40%), moderate (40%<DOS<60%) and severe (DOS>60%) as used in clinical settings for severity classification. Independent to flow rate, A̅S̅C̅_̅{̅S̅}̅ ·ASF_{RMS,S} showed statistical significant different means in mild, moderate and severe DOS grades at location 3 and 4 combined. Also, a monotonic increase was observed in the A̅S̅C̅_̅{̅S̅}̅ ·ASF_{RMS,S} with increase in DOS. This result can indicate that simple threshold-based detection can be used for classification of stenosis severity. FIG. 16 illustrates XX features demonstrates good stenosis classification, allowing for differentiation between mild (DOS<40%), moderate (40%<DOS<60%) and severe (DOS>60%) stenosis flow levels between 700 and 1200 mL/min.

### B. Stenosis Location Detection Based on Channel-to-Channel Acoustic Centroid Shift

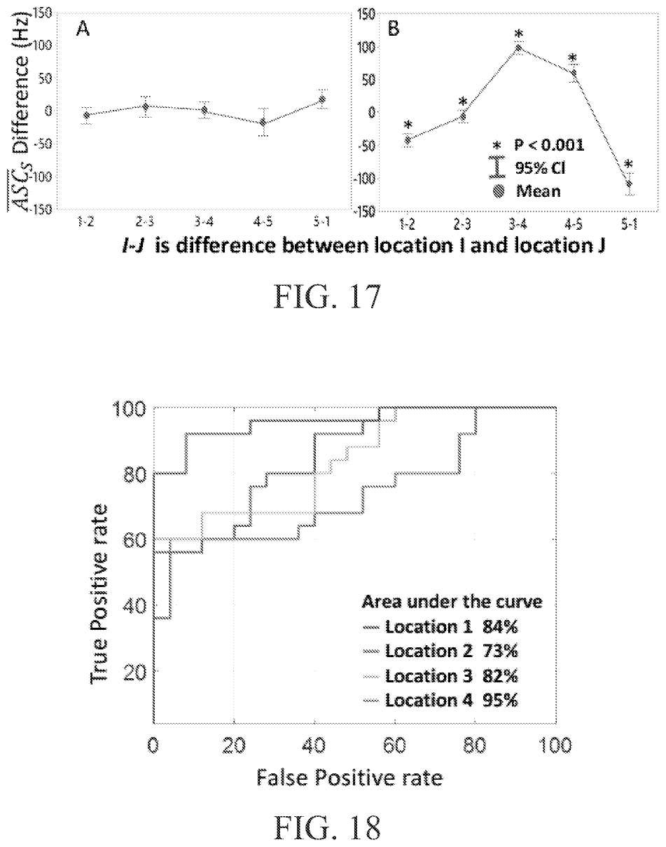

The difference in A̅S̅C̅_̅{̅S̅}̅ between adjacent locations can be calculated to estimate the most likely location of the stenosis. The interval plot, shown in FIG. 17 indicates that there is a positive shift between A̅S̅C̅_̅{̅S̅}̅ differences from proximal to distal locations. For the illustrated data in FIG. 17, the actual stenosis was located directly under location 2; location 1 was recorded 1 cm proximal, and locations 3, 4 and 5 were 1, 2 and 3 cm distal to stenosis. In general, differences between locations 3 and 4 and 4 and 5 were positive by at 50-70 Hz while the other site differences were negative. This can indicate that a simple threshold difference of 70 Hz in A̅S̅C̅_̅{̅S̅}̅ between adjacent array recording locations can identify stenosis proximally to the recording sites within a range of 1-2 cm.

### C. Threshold-Based Classifier for Point-of-Care Detection of Failing Vascular Access

For point-of-care use, a simple scheme can be used to identify which patients have a failing vascular access or are at risk for thrombosis. This can identify patients with a hemodynamically-significant stenosis, defined clinically as DOS>50%. Based on ASC and ASF values discussed above, a binary classifier was designed to classify DOS into two groups (Table V). Such patients might be selected for an imaging study or entered into a vascular surveillance program to reduce emergency interventions or for treatment planning.

TABLE V
BINARY CLASSIFIER FOR QUANTIFYING DOS SEVERITY

| Degree of stenosis | | Target |
|---|---|---|
| (DOS) | Type | vector output |
| Below 50% | Normal (healthy) | 0 |
| Above 50% | Pathological (unhealthy) | 1 |

Classifier performance can be measured by receiver operating characteristic (ROC). For each selected threshold of detection, true positive can be counted when the recording feature exceeded the threshold with DOS greater than 50%. Similarly, detection of DOS less than 50% can be classified as true negative detection. The classifier was tested on A̅S̅C̅_̅{̅S̅}̅ , ASF_{RMS,S}, A̅S̅C̅_̅{̅S̅}̅ -A̅S̅C̅_̅{̅D̅}̅, and A̅S̅C̅_̅{̅S̅}̅ ·ASF_{RMS,S} independently for each recording location.

Threshold optimization can be performed by maximizing Youden's Index (J), which is a function of sensitivity (q) and specificity (p) and is a commonly used measure of overall diagnostic effectiveness. It is defined as

$$J = max \{ sensitivity ( c ) + specificity ( c ) - 1 \}$$

over all threshold points c. The optimum threshold J₀ for J can be calculated for each classifier and for each location.

Referring to FIG. 18, the A̅S̅C̅_̅{̅S̅}̅ feature had best sensitivity at Location 2 and best specificity at Location 4. Location 2 was recorded directly over the point of stenosis and Location 4 was at the turbulent flow vortex. A similar trend was seen for A̅S̅C̅_̅{̅S̅}̅ − A̅S̅C̅_̅{̅D̅}̅. Overall, both ASC features performed well with high sensitivity (68-92%). The ASF_{RMS,S} feature had good specificity at locations distal to stenosis. Sensitivity and specificity were generally inversely proportional in nature, i.e., when sensitivity increased, the specificity decreased. Thus A̅S̅C̅_̅{̅S̅}̅ ·ASF_{RMS,S} which is the product of A̅S̅C̅_̅{̅S̅}̅ (high sensitivity) and ASF_{RMS,S} (high specificity) achieved good overall sensitivity and specificity at Location 4. This combined feature classified DOS>50% with an accuracy of 90%, specificity of 92%, and sensitivity of 92%. Best achieved area-under-the-curve was at Location 4 with 95%.

### Testing and Uses for Disclosed Embodiments

Patients with end-stage renal disease (ESRD) have significant mortality, co-morbidity, and hospitalizations compared to the general population. Longitudinal recordings of PAGs can be therefore difficult to interpret due to uncontrollable factors. There is also wide variation in vascular access anatomy in patients with ESRD, especially with malformations such as pseudo-aneurysm. To study PAGs in a controlled setting, vascular stenosis phantoms were tested with controlled degree of stenosis and variable physiologic blood pressure and flow. PAG spectra from the tuned phantoms were 98% spectrally matched to the aggregate PAG spectrum from humans with ESRD. This controlled model enabled precise description of how certain acoustic features of PAGs are affected by degree of stenosis. Further, with a priori knowledge of stenosis location, recording arrays can detect local regions of turbulent blood flow. Most importantly, statistical analyses for each DOS span a wide range of blood flow rates (700-1,200 mL/min).

Consideration must be given to how these sensor arrays would be used in the clinic or patient's home. Optionally, two-dimensional arrays can be used to cover the full vascular access anatomy. The disclosed fabrication methods support development of larger, conformal, 2-dimensional arrays to cover the full vascular access anatomy. These arrays can be bonded to removable vinyl cuffs (such as are used for blood pressure measurement) and wrapped around the vascular access during recording to maintain pressure on all microphone sites. Arrays can be flexible enough to bend around 0.6-mm radius curves at 90 degrees. Microphones can require only 50 mN of force against the skin for accurate recording. Additionally, the array materials exposed to skin (polyimide and silicone) can be wearable applications without causing skin allergy. As larger arrays are fabricated, acoustic bandwidth matching between channels can be addressed, as large changes in microphone frequency response could affect spectral analysis. Clinical testing with larger arrays is needed to determine how well these constraints can be met in human use.

Thresholds could be used for clinical monitoring of stenosis formation. Site-to-site differences in systolic ASC greater than 70 Hz can be caused by the presence of vascular stenosis. In terms of stenosis classification, a simple threshold-based binary classifier based on a combined feature can be determined. Compared with more complex training-based classifiers, a simple threshold-based classifier is potentially more generalizable (e.g. to develop clinical standards for interpretation), and less susceptible to over-fitting. Additionally, because the ASC and ASF calculations average out spurious peaks, a simple threshold can be sufficient for clinical use. In further aspects, use of binary threshold can be too simplistic for complex cases. Therefore, clinical testing can be used to determine how robust the detection is to false positives due to motion artifacts or insufficient sensor contact pressure.

### Optional Sensor Aspect Characterization

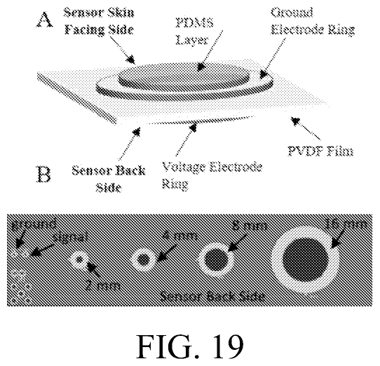

In some aspects, sensors 102 having various diameters (e.g., diameter of the hole 110 through the electrodes) and outer layers 126 can be characterized. For example, sensors having diameters of 2 mm, 4 mm, 8 mm, and 16 mm were characterized. Further, multiple outer layers were characterized, including air, ECOFLEX 00-10 PDMS film, and DOW CORNING SYLGARD 527 dielectric silicone gel. FIG. 19 illustrates a test board in accordance with the characterization tests.

For said characterization, the sensors were tested under the same conditions to determine the best combination of sensor size and backing material, and their responses were compared to a conventional stethoscope. Characterization tests included slow frequency sweeps to determine the acoustic frequency response and single-tone tests to determine SNDR. Functional tests involved signal recordings from a vascular flow phantom to simulate the recording of hemoacoustics. Sensor data was collected using LabVIEW at sampling rate of 10 kHz. Sensor data was compared to data collected from a digital recording stethoscope (3M Littmann Stethoscope 3200). The sampling rate of the stethoscope was 4 kHz, but this was digitally upsampled to 10 kHz to match the sample rate of the PVDF sensors for frequency analysis.

### A. Frequency Response

A frequency generator was used to generate a linear frequency sweep from 20 Hz to 5 kHz over 60 sec. The output was connected to a contact speaker element to generate acoustic vibrations through a 6 mm layer of PDMS rubber. This arrangement mimicked the typical thickness of tissue over a blood vessel in a vascular access. To account for variations in surface coupling pressure and gain differences, recordings from sensors and the stethoscope were normalized to a −10 dB RMS level in MATLAB. Power spectral densities from this test revealed the frequency responses of the PVDF sensors and the stethoscope. Generally, PVDF sensors had a flatter frequency response and wider bandwidth.

To compare the relative frequency response of PVDF sensors to the stethoscope, the power spectral density of the latter was subtracted from each sensor response to normalize the sensor response to that of the stethoscope. From this analysis several effects were evident. First, the PVDF sensors had relatively less low-frequency response when compared to the stethoscope, with an average 0-dB crossover at approximately 30 Hz. Second, they had significantly greater sensitivity in the range of 30-300 Hz, and also above 700 Hz. Finally, of the PVDF sensors, those with silicone gel backing had an additional 3 dB gain in most frequency ranges. FIG. 20 illustrates a frequency response comparison between sensors with different backing layers (A-C). When plotted relative to the stethoscope response, OVDF sensors showed enhanced mid-band and high frequency gain (D-F).

### B. Single Tone Response

Single tone testing was done at 3 frequencies (150, 300, and 450 Hz) using the same setup (FIG. 8A). To compare the response between sensors and the stethoscope, the SNDR was calculated (Table VI). On average, all PVDF sensors outperformed the stethoscope, with the 2-mm sensor plus silicone gel backing showing the best overall SNDR.

TABLE VI
Measured SNDR of PVDF Sensors and Stethoscope at 150, 300, and 450 Hz

| Frequency | | PDMS Backing | | | Silicone Gel Backing | | | No Backing | | |
|---|---|---|---|---|---|---|---|---|---|---|
| (Hz) | Stethoscope | 8 mm | 4 mm | 2 mm | 8 mm | 4 mm | 2 mm | 8 mm | 4 mm | 2 mm |
| 150 | 11.9937 | 20.3000 | 33.2125 | 23.3171 | 16.1953 | −2.1913 | 30.2868 | 1.9916 | 12.4593 | 10.9727 |
| 300 | −0.6459 | 27.7621 | 7.2581 | 31.5992 | 26.6204 | 23.0329 | 33.7264 | 20.2204 | 16.7918 | 26.6825 |
| 450 | 16.3006 | 28.5185 | 12.5628 | 30.8861 | 31.5639 | 9.6512 | 34.5464 | 21.0196 | 28.9440 | 26.4480 |

### C. PVDF and Stethoscopic Recording of Hemoacoustics

To simulate how the sensor would perform on a real patient (but in a controlled environment), recordings were made on a vascular access phantom. The phantom comprised a 6-mm silicone tube embedded in PDMS (Ecoflex 00-10) at a 6-mm depth. Stenosis was simulated in the center of the phantom with a band tied around the tube to produce an abrupt narrowing.

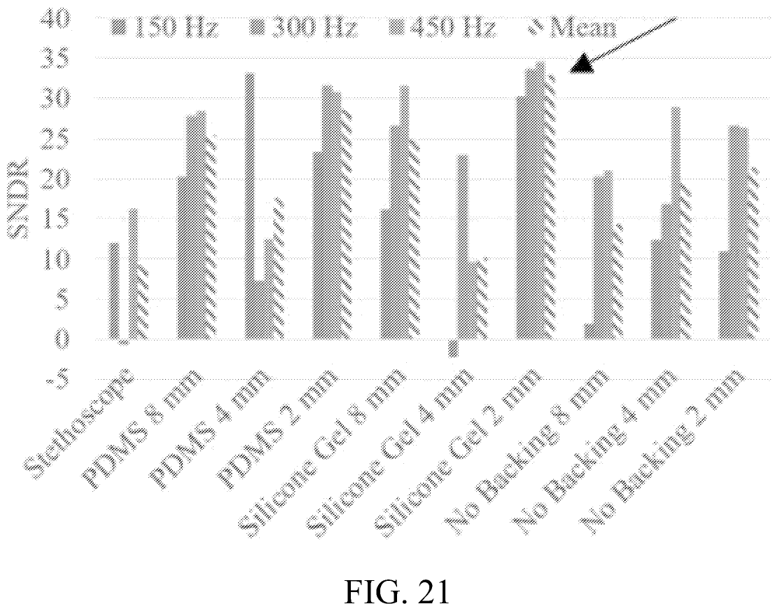

The phantom was connected to a pulsatile flow pump to simulate human hemodynamics at 432 ml/min and 1120 ml/min (low and high flow rates, respectively). The 2-mm PVDF sensor with silicone gel backing showed similar signal recording quality to the stethoscope until 100 Hz. At higher frequencies the stethoscope was relatively less sensitive. FIG. 21 illustrates sensors with 2 mm diaphragms had the highest SNDR and performed well at all three test frequencies.

FIG. 21 illustrates signal comparisons between sensors having different sizes and backings at multiple frequencies.

### D. Autoregressive Modeling of PAG Systoles

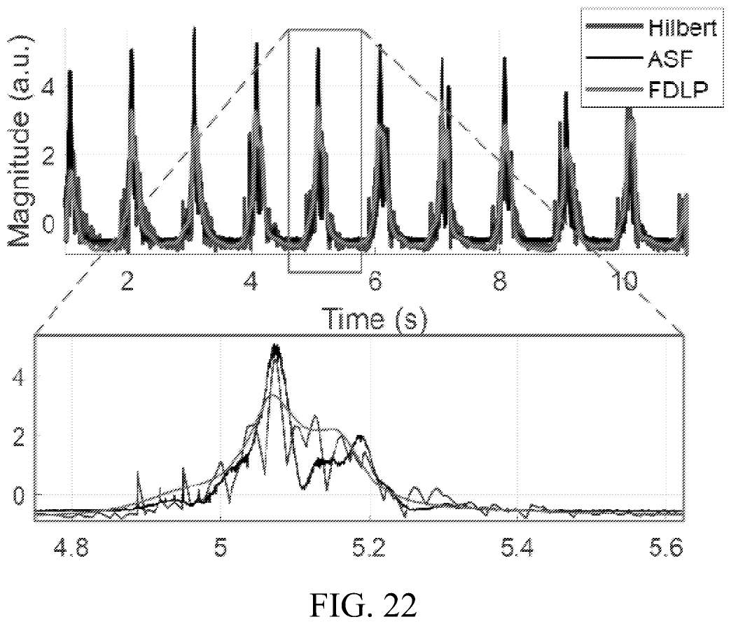

FIG. 22 illustrates analytical signals obtained from PAGs can include Hilbert envelope, auditory spectral flux, and FDLP-modeled envelopes. Of these signals, FDLP produced the smoothest response, permitting efficient feature extraction.

When applied to PAGs, FDLP can be used for systolic pulse enhancement, or to produce an analytic signal for feature extraction, e.g. to estimate flow variations. Here, the FDLP envelope can be applied as a systole enhancement filter by multiplying the envelope by the original PAG signal to apply time-based signal shaping.

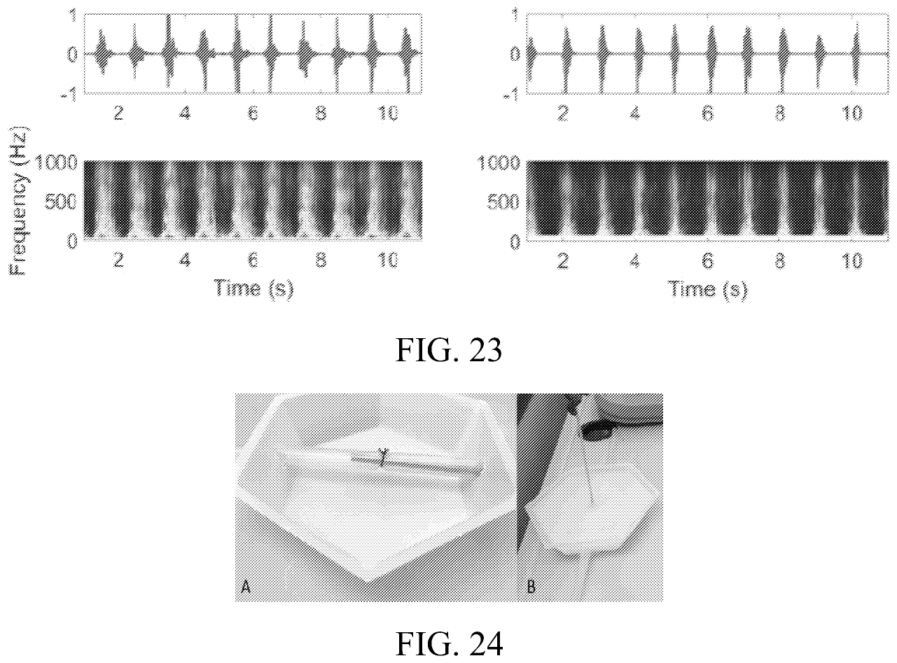

Consider recordings from the 2-mm, silicone gel sensor processed using the FDLP systole enhancement compared to a conventional stethoscope recording from the same phantom processed with the same FDLP systole enhancement filter. Spectrograms were computed over 6 octaves with 12 voices/octave, starting at scale 3. The combination of the improved frequency response of the 2-mm sensor and the FDLP processing enhances the systole dramatically and reduces inter-systole noise. FIG. 23 illustrates time and wavelet scale representations of PAG recorded from (A) conventional stethoscope, and (B) the 2 mm sensor with silicone gel, both using FDLP systole pulse enhancement.

It can be shown that PVDF sensors acoustic response is approximately 10 dB/decade lower than stethoscope acoustic response. However, in the 150-450 Hz range, single-tone testing revealed that PVDF sensors had generally better SNDR. The former also have a wide and relatively flat frequency response to 5 kHz, although the stethoscope acoustic response is likely limited by built-in signal processing to reduce noise pickup. Overall, it can be concluded that a 2-mm PVDF diaphragm with silicone gel backing forms a reliable transducer for skin-coupled recording of PAGs. In addition, the proposed construction method can be easily modified for use with flexible, polyimide printed circuits to enable flexible arrays of skin-contact microphones. These array microphones can enable point-of-care monitoring of vascular access and can leverage multi-channel signal processing for rejection of interference and new PAG analysis features.

### Vascular Access Phantom

Because the broad population of hemodialysis patients vary greatly in terms of flow types, flow rates, and degree of stenosis (DOS), a vascular stenosis phantom was developed to mimic human physiology. This phantom allowed independent control of hemodynamic parameters and DOS to reduce the variability in signal analysis to aid feature identification. Phantoms were developed assuming common targets for vascular access in humans: a 6 mm blood vessel, 6 mm vessel diameter, and nominal flow rate of at least 600 mL/min. Vascular stenosis phantoms were made using 6 mm silicone tubing and bio-mimicking silicone rubber (Ecoflex 00-10). A double band suture was tied around the tubing in the center of the phantom to produce turbulent blood flow as predicted by vascular blood flow simulations. The DOS was controlled by tying the suture around metal rods with fixed diameters (FIG. 24A). After suturing, liquid silicone rubber was poured around the vessel phantom and the vessel depth was controlled using a height adjustment jig while the silicone cured (FIG. 24B). FIG. 24A-B illustrate steps in the fabrication of a vascular stenosis phantom using a suture band around silicone tubing and cast into a bio-mimicking silicone rubber mold.

Two pulsatile pumps (e.g., Cole Parmer MasterFlex L/S, Shurflo 4008) (FIGS. 13A-B), were used to create different hemodynamic flows of blood-mimicking fluid through the phantoms at 60 beats per minute.

TABLE VII
Pulsate Pump Combinations

| Flow | | Flow Rate |
|---|---|---|
| Number | Flow Type | (mL/min) |
| 1 | Monophasic | 312 |
| 2 | Biphasic | 97 |
| 3 | Biphasic | 242 |
| 4 | Biphasic | 395 |
| 5 | Biphasic | 404 |
| 6 | Biphasic | 541 |
| 7 | Biphasic | 566 |
| 8 | Biphasic | 695 |
| 9 | Biphasic | 722 |
| 10 | Biphasic | 764 |
| 11 | Biphasic | 843 |
| 12 | Biphasic | 978 |
| 13 | Biphasic | 1002 |

Thirteen flow types, described in Table VII, were used to span the range of flows found in human vascular accesses. Pulsatile pressures and aggregate flow rate were measured with a flow sensor (Omega FMG91-PVDF) and a pressure sensor (PendoTech N-038 PressureMAT). In one exemplary study, only flows from 500-1,010 mL/min were analyzed to represent the nominal flow range in functional vascular accesses. [00188]10-second PAGs were recorded at three locations on each phantom with a digital recording stethoscope (Littman 3200) and used in signal analysis.

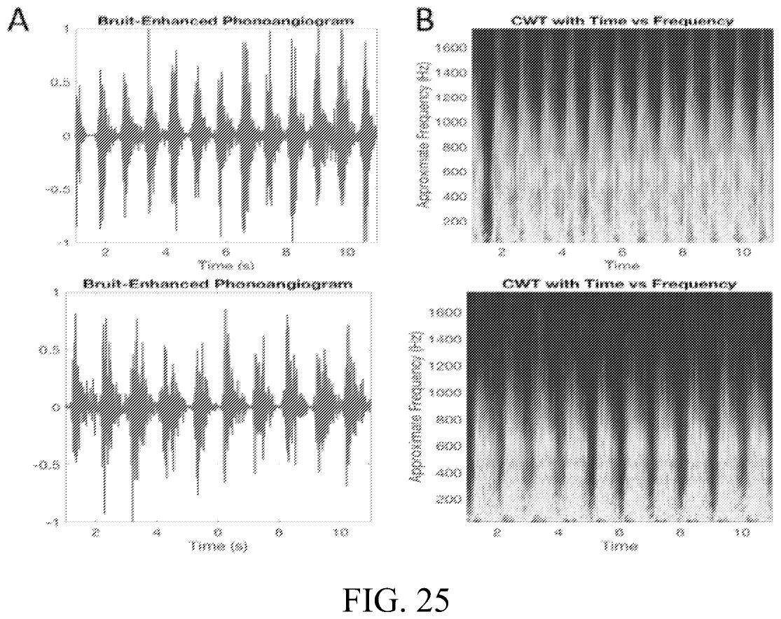

FIG. 25 illustrates phonoangiogram (A) and wavelet transform (B) of typical dialysis patient and vascular phantom without stenosis. The phantom produces similar blood flow sounds, but with a more repetitive flow pattern and pulse rate.

Recordings from the vascular phantom were compared to those from humans to ensure that they were physiologically relevant. An aggregate power spectrum was produced from 3,283 unique 10-s recordings obtained from 24 hemodialysis patients over 18 months. Spectral comparisons between human and phantom data were used to reduce time-domain variability e.g. due to heart rate differences (FIG. 25A). Human and phantom spectra followed similar spectral trends and showed similar dynamic range in time-domain analysis (FIG. 25B). Quantitatively, normalized RMS error of the aggregate power spectrum (FIG. 14C) was calculated over the aggregate human PAG spectra range. The vascular phantoms matched the aggregate human PAG spectra with a 1.84% normalized RMS error, scaled to the total power spectrum range. This comparison suggested that the vascular stenosis phantoms can adequately replicate PAGs as measured from humans, but with control over vascular access flow rate and stenosis severity.

Fourteen spectral features were calculated for each PAG (Table VIII). All features were calculated in Matlab software based on ten-second PAG recordings from vascular phantoms.

TABLE VIII
Phonoangiogram Features

| Feature | Flow Type |
|---|---|
| 1 | Average of ASC |
| 2 | Average of ASF |
| 3 | RMS of ASC peaks |
| 4 | RMS of ASC peak widths |
| 5 | RMS of ASF peak widths |
| 6 | RMS of ASC peak prominence |
| 7 | RMS of ASF peak prominence |
| 8 | Time-aligned ratio of ASC to ASF |
| 9 | ASC values at ASF peak |
| 10 | Variance of ASC |
| 11 | Variance of ASF |
| 12 | Covariance of ASC and ASF |
| 13 | Correlation corresponding to the |
| | covariance matrix of ASC and ASF |
| 14 | Standard deviations of the correlation |
| | matrix (Feature 13) |

Features were derived from two analytical approaches: continuous wavelet transform and autoregressive linear predictive coding. Wavelet coefficients were computed using the Morlet wavelet; wavelet scales were computed over 6 octaves with 12 voices/octave, starting at scale 3. From the wavelet space, the Auditory Spectral Centroid (ASC) was calculated. ASC is the weighted mean of the frequencies present in the signal. The ASC value is calculated using

$$ASC = \frac{{\sum }_{n = 0}^{N - 1} f ( n ) x ( n )}{{\sum }_{n = 0}^{N - 1} x ( n )} ,$$

where x(n) is the weighted frequency value of bin number n, and f(n) is the center frequency of bin number n.

Autoregressive modeling was used to produce an analytic signal similar to a Hilbert envelope, but derived from the discrete cosine transform. By fitting a linear predictive model to the frequency dual of the PAG, a smooth approximation of the time-domain power envelope was obtained. This analytical envelope is highly correlated to a common measure, Auditory Spectral Flux (ASF), which describes how quickly the power spectrum of a signal is changing. Because the modeled analytical envelope was smoother, it was used as a surrogate for ASF in feature extraction.

Seven features were derived from the ASC and ASF equivalent signals: mean value, rms value of ASC peaks, rms value of ASC and ASF peak widths, and rms value of ASC and ASF peak prominence. Seven multi-variate features were also calculated: correlation and covariance between ASC and ASF, the time-aligned ratio of ASC to ASF, and the corresponding ASC value at ASF peaks.

### A. Stenosis Feature Correlation Plots

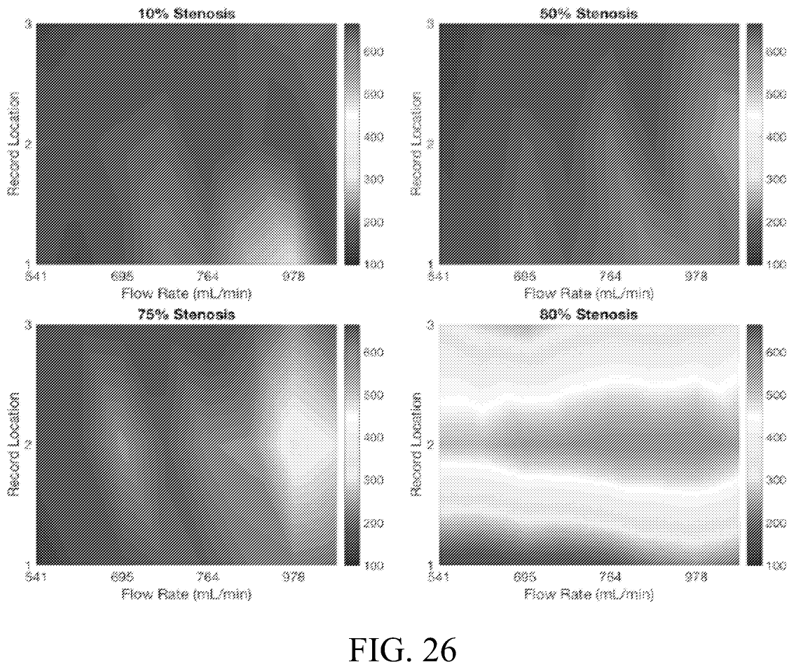

Qualitative analysis of each computed feature was performed using heat maps for each stenosis phantom, combining variability in recording location and hemodynamic flow rate. All maps showed feature trends dependent on flow type and recording location. FIG. 26 illustrates ASC values at different flow types and recording locations for phantoms with DOS between 10-80%. Of the 14 features, the mean ASC value showed the clearest trend at the site of stenosis across the different phantoms.

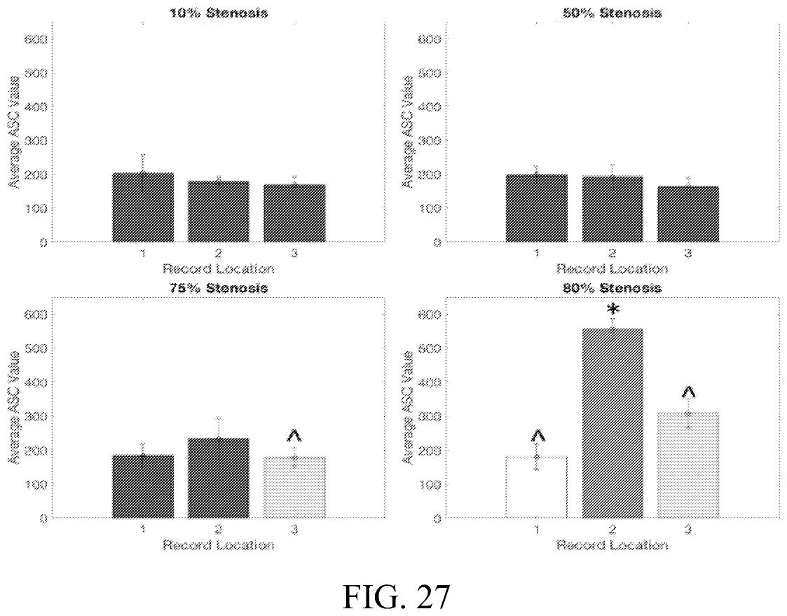

FIG. 27 illustrates the average ASC value across physiologic flow levels at all three recording locations for phantoms with DOS between 10-80%. \hat{ }p<0.05 for Location 1 and Location 2 or Location 2 and Location 3. * p<0.05 for all three locations. Because mean ASC value correlated strongly with DOS, its performance can be characterized by localizing and describing stenosis level. Many of the analyzed features, however, produce similar outcomes. To mimic a clinical monitoring scenario, in which patient blood flow rate varies according to anatomical and physiologic factors, all observations at a flow rate greater than 500 mL/min were pooled for statistical analysis. Paired t-test comparisons were used to determine statistical significance between mean ASC values at each recording site and for each stenosis phantom.

ASC values across the three recording locations were not statistically significant at lower DOS (below 50%). At 60% DOS, a statistically-detectable shift between recording locations first became apparent (p=0.0018). Above 80% DOS, ASC values at all recording locations were statistically distinct (p=2.37e-09). The differentiation of the average ASC value at each location suggests that signal processing of PAGs can be used to localize vascular stenosis.

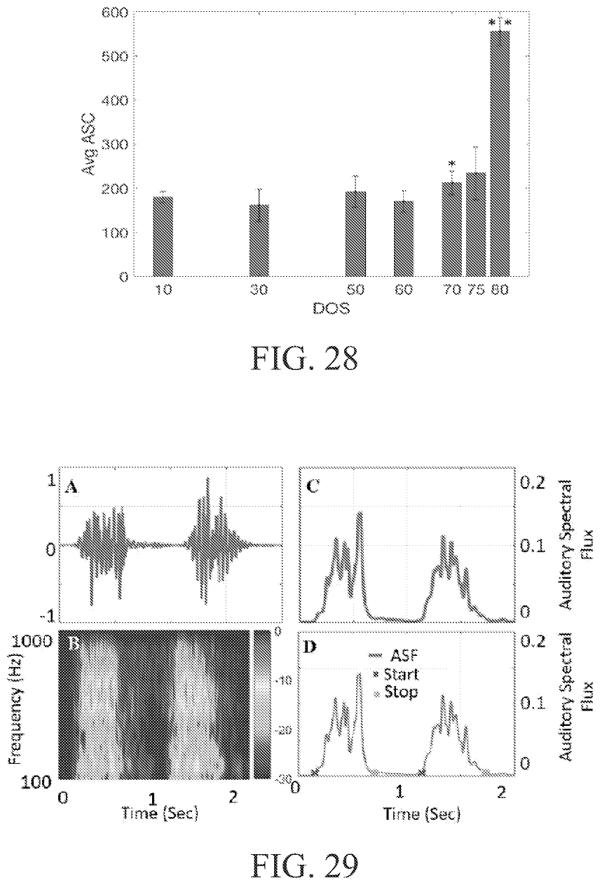

To determine how well mean ASC value can describe the level of stenosis, recordings from only Location 2 for each phantom were analyzed. FIG. 28 illustrates mean ASC values for all flow types recorded at Location 2 for each phantom. A statistically-significant shift was observed for each phantom above 60%. Phantoms below 60% showed no discernible trend in mean ASC value. Beginning with a DOS at 60%, a monotonic increase in mean ASC value was observed as the DOS increases. Specifically, the mean ASC value increased from 169.83±24.48 Hz at 60% stenosis to 555.92±31.21 Hz at 80% stenosis. This nonlinear trend implies that the ASC can estimate DOS with increasing specificity above 60% stenosis.

### Example Data Analysis

FIG. 29 illustrates time-domain blood sound (A) and CWT spectral domain (B) collected from a phantom, as disclosed herein, using a sensor array, as disclosed herein. (C) illustrates extracted analytic signal Auditory Spectral Flux, and (D) illustrates systolic start/end times calculated by thresholding.

As disclosed herein, ASF can approximate a spectral first derivative of the signal and so estimates the temporal-spectral envelope of the signal since it indicates regions of high flux between coefficients of the CWT. Although it appears similar to the Hilbert envelope, ASF is calculated directly from CWT coefficients and not by inverse reconstruction, therefore it is not an analytic signal.

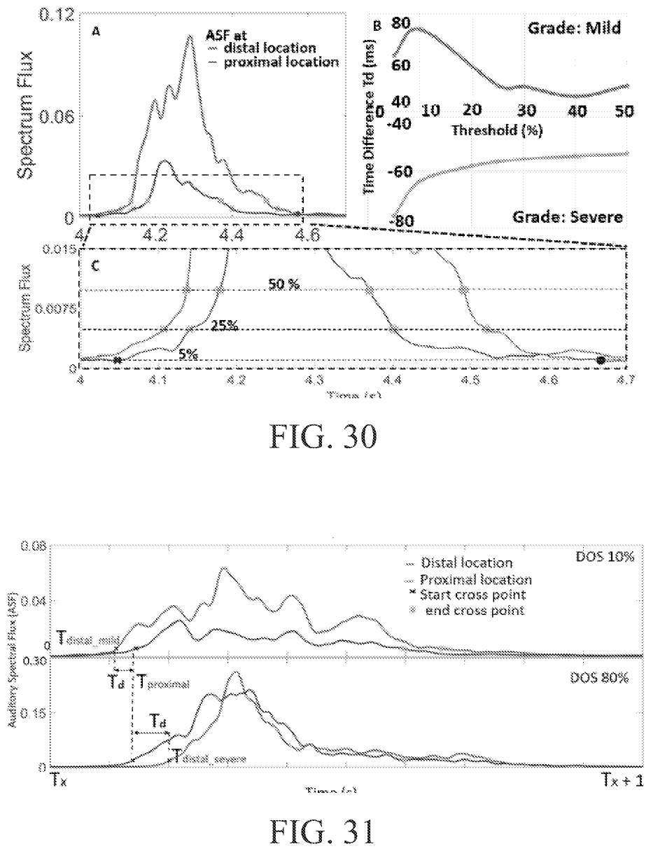

Thresholding of ASF can be performed to identify systolic and diastolic phases. Identification of these phases can be used for blood velocity calculation, because flow acceleration only occurs on the high-pressure systolic pulse. Threshold selection was performed at varying percentages of the ASF RMS value. Optimization required a tradeoff between rejecting ASF spurs in the diastolic period (higher threshold) and maximizing systolic pulse width for improved velocity accuracy (lower threshold). A threshold of 25% of ASF RMS was chosen to balance these tradeoffs (FIG. 30).

The 25% of ASF RMS threshold was calculated for each recording to detect onset and end of the systole. This level was chosen empirically from data recordings to provide reliable pulse detection. The pulse width was calculated as the difference between the start and end of the pulse. This width was used for filtering to reduce the chances of faulty cross-point detection and other artifacts in two stages. In the first stage, longest width was calculated from all the pulses while in the second stage 40% of that width was chosen as the minimum width criteria and 1 second was chosen as the maximum based on established systolic segmentation approaches.

### Temporal-Spectral Feature Extraction

The stenosis can be classified from the data collected. According to one aspect, features can be shown from locations taken 1 cm proximal to a stenosis and 2 cm distal to the stenosis. These locations can be selected based on the presence of turbulent flow, as described herein.

Referring to FIG. 31, time differences between ASF threshold crossing, T_{d}, can be calculated as the difference between cross-point at proximal location (Tₚᵣₒₓᵢₘₐₗ) and cross-point at distal location, i.e. T_{d}=Tₚᵣₒₓᵢₘₐₗ−T_{distal}. FIG. 31 illustrates that ASF calculated at proximal and distal locations show an inversion in T_{d}. At moderate and severe DOS, T_{d} can be negative, indicating flow velocity increase.

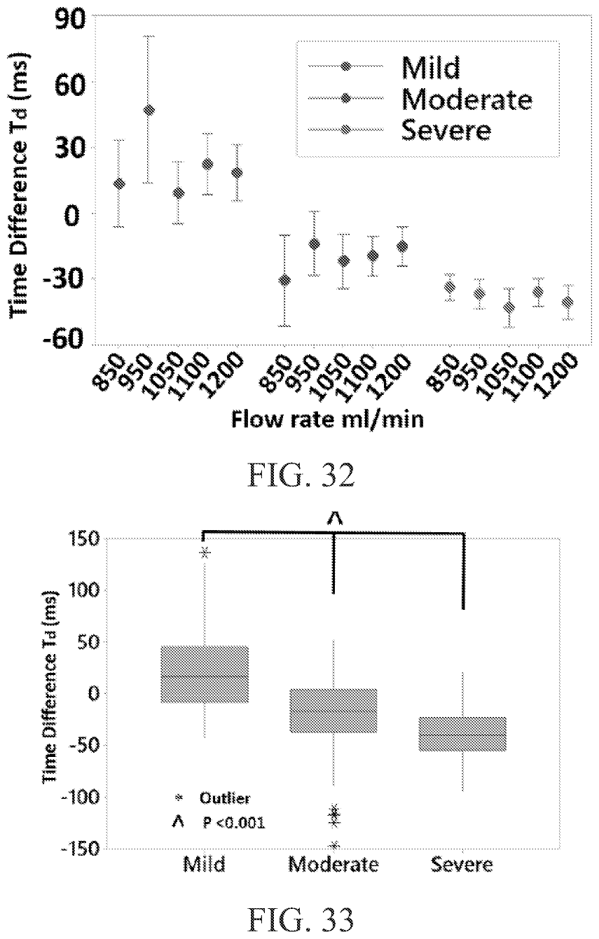

T_{d} can be measured over the range of all the physiological flow rates and it can be relatively intolerant to flow rate, as illustrated in FIG. 32. This suggested that DOS and blood velocity change could be calculated independent of flow rate, which varies between patients. FIG. 32 illustrates T_{d} over the range of flow rates and DOS grade. T_{d} dependence on flow can be low, suggesting that DOS can be assessed independently of patient-variable flow rates.

FIG. 33 illustrates time differences for each DOS grade. Stenosis classification based on T_{d} can be performed by grouping tested phantoms into 3 grades of DOS: mild (DOS<40%), moderate (40%<DOS<60%) and severe (DOS>60%). Time differences can be unique between all groups (p<0.001, n=50, balanced ANOVA) and show a monotonic behavior, as seen in FIG. 33, depending on DOS. For hemodynamically significant stenoses (moderate and severe classes), an inversion in T_{d} can be seen, such that distal sites can show systolic ASF increase before proximal sites.

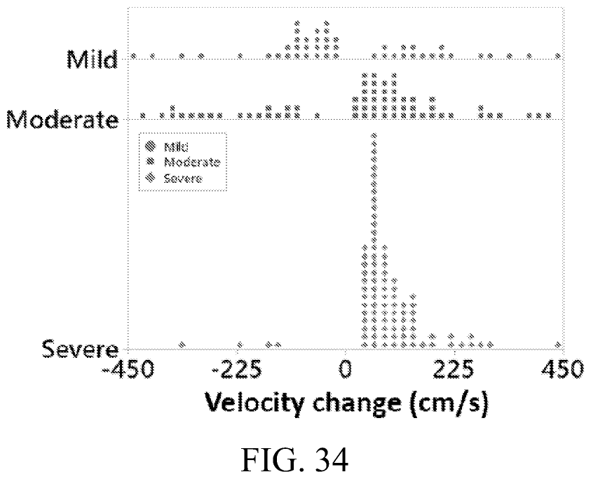

Referring to FIG. 34, showing mean velocity change for each DOS grade, to quantify the flow acceleration, velocity increase can be calculated as the distance between the recording sites (3 cm) divided by the T_{d}. In one dataset, over all ranges of flow and DOS, mean calculated velocity increase was 142 and 155 cm/s from mild to moderate and severe DOS. This result was within the expected physiological range for blood velocity in 6-mm caliber vessels. For stenoic phantoms with DOS>40%, an average increase of 142 to 155 cm/s was calculated from blood flow acoustic data.

Accordingly, it can be seen that the time difference T_{d} in the onset of ASF in each systolic pulse can become inverted in the presence of hemodynamically significant stenosis. Accordingly, a threshold of T_{d}<0 ms can be used as a screening criterion for significant stenosis in clinical monitoring.

### Example 2

### Signal Processing: Considerations in the Transduction of Bruits

This section provides certain design considerations for a transducer and front-end interface amplifier to best capture the relevant acoustic signals to the accuracy needed for classification.

### Skin-Coupled Recording Microphone Array

The true spectral bandwidth and dynamic range of vascular sounds may still be unknown since only stethoscopes have been used to record these signals previously. Published analyses of PAGs report higher-pitched sounds associated with vascular stenosis, which suggests that the reduced frequency range of stethoscopes can be insufficient for blood sounds. Therefore, acoustic recordings from the in vitro phantom were made with a reference transducer (Tyco Electronics CM-01B) with a flat frequency response to at least 2 kHz. For each recording, the 95% power bandwidth was calculated by integrating the power spectral density. To compute the power bandwidth, the power spectral density was computed using Fast Fourier Transform, then cumulatively integrated by frequency bin until the integration met 95% of the total power in all bins. Because electronic circuits suffer from increased flicker noise at low frequencies, and because all prior reports of PAGs indicate increased power above 100 Hz associated with vascular stenosis, a lower integration bound of 25 Hz was adopted. This had a further benefit of enabling shorter-duration recordings (e.g. 10 seconds), which otherwise do not accurately capture extremely low-frequency signal components. For this analysis 10-second recordings were taken 1 cm before the simulated stenosis, at the stenosis, and 1 and 2 cm after the stenosis relative to the direction of blood flow.

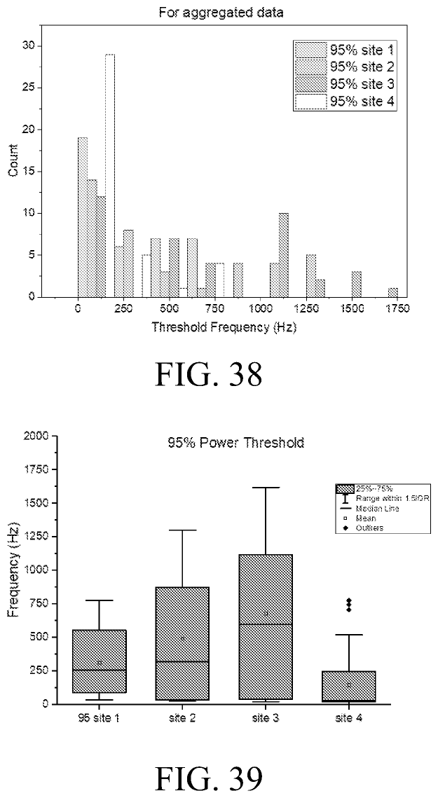

Signal bandwidth was related to the degree of stenosis, as expected, but also to recording location, as shown in FIGS. 37-38. Both effects were expected based on prior measurements and simulations indicating turbulent flow existing up to 1-2 cm from a typical stenotic lesion. These results suggest the benefit of recording from multiple locations to accurately detect the presence and severity of a stenotic lesion. In an analysis of 156 recordings, the maximum interquartile range for 95% bandwidth was 25 Hz-1.2 kHz; the lower frequency bound correlated with phantoms with low DOS producing little turbulent flow, as shown in FIG. 39. These data suggest that a signal bandwidth of at least 1.5 kHz is appropriate for measuring vascular bruits. A bandwidth of 25-2.25 kHz was adopted (with a safety factor).

The required bandwidth was achieved with a signal-to-noise ratio of 24 dB using a polyvinylidene-fluoride (PVDF) film as a 2-mm diameter circular transducer. This transducer was developed to be coupled directly to the skin to measure blood sounds through direct piezoelectric transduction. The small size of the transducer allowed it to be fabricated in recording arrays.

### Transducer Front-End Interface Amplifier Design

Each PVDF microphone in the recording array can be coupled to an interface amplifier to amplify the signal amplitude before digital conversion. The analog performance of the interface amplifier can be driven by three constraints: the electrical impedance of the PVDF transducer, the required signal bandwidth, and the required dynamic range. In exemplary aspects, the dynamic range constraint can be driven by the minimum signal accuracy needed for the digital signal processing and classification strategy. In an exemplary retrospective analysis of blood sounds measured from hemodialysis patients and an in vitro phantom, it can be determined that a minimum dynamic range of 60.2 dB was needed for accurate classification of stenosis severity, which is roughly equivalent to 10-bit accuracy after digital conversion. As described in the previous section, a bandwidth of 2.25 kHz is needed to capture most of the energy in the PAG signals.

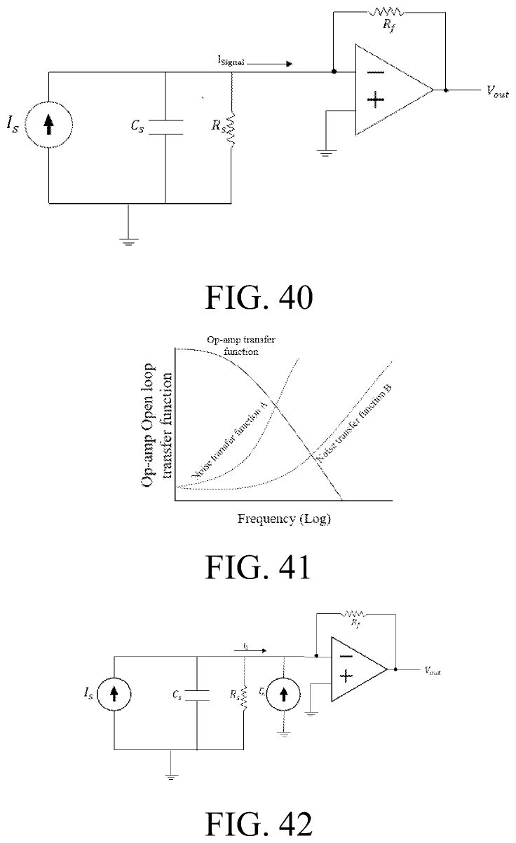

The amplifier input impedance constraint is based on the electrical model for each 2-mm transducer which was extracted using an impedance analyzer (Hioki IM3570). The PVDF transducer was modeled electrically as a resistor and capacitor in parallel, shown in FIG. 40. Measured values of the sensor resistance, capacitance, and the equivalent sensor output current when recording PAGs are shown in Table 1. Rₛ and Cₛ can be functions of properties of the sensors, such as the materials used and dimensions of the sensors.

TABLE 1
Measured transducer parameters for an
electrical model for the PVDF Sensor

| Parameter | Nominal Value | |
|---|---|---|
| Current Source Amplitude (I_{signal}) | 0.63 | μA |
| Current Source Frequency | 100 | Hz |
| Sensor Resistance (R_{S}) | 12.4 | MΩ |
| Sensor Capacitance (Cₛ) | 100 | pF |

Because the PVDF transducer can have a large impedance with a small signal current, a transimpedance amplifier (TIA) can be used to convert the piezoelectric sensor current to a voltage that can be digitized. Each microphone within the array can feed a dedicated TIA. The TIA can convert the current produced by the transducer to an output voltage while minimizing the input referred noise power. The TIA can be an ideal interface to high impedance, current output devices, but certain critical design considerations can be made to optimize the total signal-to-noise ratio of the output signal. An important design consideration, which has a direct impact on the sensitivity, is the input-referred noise of the TIA. In feedback TIAs built using general voltage amplifiers such as an op-amp with a shunt-shunt feedback, the input referred noise is a function of the input-referred voltage and current noise of the op-Amp. Therefore, op-amps with high input-referred voltage (nV/\sqrt{Hz}) and/or current noise (nA/\sqrt{Hz}) can optionally be avoided.

The design specifications for the TIA were chosen assuming it would be followed by a 2nd-stage programmable gain amplifier and a 10-bit analog-to-digital converter. Therefore, a small-signal output level was chosen to limit harmonic distortion which can occur with large signal swing. The performance of the TIA dominates the analog noise floor and linearity, so these later stages are not described here. Design requirements for the TIA are summarized in Table 2 based on measured properties from PAGs in humans and the vascular phantom.

TABLE 2
Design Specifications for Transimpedance Interface Amplifier

| Parameter | Nominal Value |
|---|---|
| Nominal input current: | 0.63 μA (from PVDF transducer) |
| Required dynamic range | 60.2 dB minimum |
| Nominal output voltage level | 30 mV peak |
| Low-pass signal bandwidth | 2.25 kHz |

In addition to the inherent noise of the op-amp, the feedback resistor can play a role in the overall input-referred noise power of the TIA. Increasing the feedback resistance can not only reduce the noise current associated with the resistance but also result in higher TIA gain, which can help lower the overall input-referred noise of the TIA. Nevertheless, the requirement imposed on the frequency response of the TIA when interfacing with the transducer, limits the amount of resistance that can be used in the feedback path. Still, optimizing the feedback resistance will lead to lower input-referred noise within the required gain bandwidth (GBW) of the TIA, as can be determined from the transfer function shown in FIG. 41. For example, the GBW can be a product of the amplifier's bandwidth and the gain at which the bandwidth is measured (e.g., the unity gain bandwidth of the amplifier).

The performance metrics are beneficial in completing the design process. Major small-signal TIA performance metrics can include the transimpedance gain, the 3-dB bandwidth, and input-referred noise power. Considering the transimpedance gain and the bandwidth, the feedback network can be the first physical parameter that is be determined. The feedback network can include a resistor and capacitor that are connected in parallel. The resistive part can help set the transimpedance gain of the TIA while the capacitive component helps with setting the frequency response, particularly the bandwidth and the stability. The frequency response can affect the TIA noise transfer function, and consequently, the input referred noise of the TIA, too. Eq. 5 and Eq. 6, which are provided below, demonstrate how to optimize feedback capacitor ranges, e.g.,

$$\begin{matrix}If ( \frac{{R}_{f}}{{R}_{s}} + 1 ) \geq 2 \sqrt{{R}_{f} * {C}_{s} * GBW} & {C}_{f} = \frac{{C}_{s}}{2 ( \frac{{R}_{f}}{{R}_{in}} + 1 )}\end{matrix} \qquad Eq . 5$$

$$\begin{matrix}If ( \frac{{R}_{f}}{{R}_{s}} + 1 ) \leq 2 \sqrt{{R}_{f} * {C}_{s} * GBW} & {C}_{f} = \sqrt{\frac{{C}_{s}}{{R}_{f} * GBW}}\end{matrix} \qquad Eq . 6$$

wherein R_{f} is feedback resistance and Rᵢₙ is the equivalent input resistance.

Another valuable consideration for feedback capacitor value is the desired cutoff frequency. This cutoff frequency can determine the TIA's −3 db bandwidth, f_{-3dB}, expressed as

$${f}_{- 3 dB} = \frac{1}{2 π * {R}_{f} * {C}_{f}}$$

Input referred power is defined by the ratio of the output noise power, divided by the TIA transfer function. This can be calculated using the signal-to-noise ratio (SNR) of the circuit, as shown in FIG. 42:

$$SNR = \frac{{Power}_{signal}}{{Power}_{noise}} = \frac{{{I}_{s}}^{2} * {R}_{f}}{\overline{{ι}_{n}^{2}} * {R}_{f}} = \frac{{{I}_{s}}^{2}}{\overline{{ι}_{n}^{2}}}$$

where iₙ is the output noise current and Iₛ is the current source amplitude.

The TIA design process is to maximize SNR given constraints on required bandwidth, available supply voltage/current, and necessary dynamic range. The transfer function of output voltage level (Vₒᵤₜ) and input current (I_{signal}) is dependent on the feedback resistance:

$${V}_{out} = - [ {I}_{signal} * {R}_{f} ] + {V}_{ref}$$

In this example, a reference voltage, V_{ref}, is generated by a voltage divider with two resistors having resistances of R₁ and R₂. Both were selected to be 10 kΩ to set the reference at half of the supply voltage V_{supply}, i.e.

$${V}_{ref} = {V}_{supply} * \frac{{R}_{2}}{{R}_{1} + {R}_{2}} ( where {R}_{2} = {R}_{1} )$$

The DC value of the output for this stage of amplification was selected to be 2.1V. From this parameter, the feedback resistance was calculated as

$${R}_{f} = \frac{{V}_{out} - {V}_{ref}}{{I}_{signal}} = \frac{2.1 V - 1.65 V}{0.63 μ A} = 715 k Ω \qquad Eq . 7$$

The value of the feedback capacitance was determined from the required signal bandwidth. Rearranging Eq. 7 for C_{f} provides:

$${C}_{f} = \frac{1}{2 π * {R}_{f} * {f}_{- 3 dB}} = \frac{1}{2 π * 715 k Ω * 2.25 kHz} = 100 ρ F$$

The minimum op-amp bandwidth f_{GBW} for this circuit was calculated using the feedback resistance and capacitance, R_{f} and C_{f}, as well as the capacitance of the input pin of the selected op-amp (Texas Instruments OPA2378). The IN-pin capacitance is the sum of the sensor capacitance (Cₛ), common-mode input capacitance (C_{CM}), and differential mode capacitance (C_{Diff}) as

$${C}_{IN} = {C}_{s} + {C}_{CM} + {C}_{Diff} = 1000 ρ F + 5 ρ F + 4 ρ F ≅ 1000 ρ F \qquad Eq . 8$$

$${f}_{GBW} \geq \frac{{C}_{f} + {C}_{in}}{2 π * {R}_{f} * {C}_{f}^{2}} \geq 24.7 kHz \qquad Eq . 9$$

Therefore, the op-amp can have a minimum bandwidth of roughly 25 kHz. The OPA2378's 900 kHz bandwidth satisfies this requirement, and is a viable component for this application. The OPA2378 has an input voltage noise density of

$$\frac{20 nV}{{Hz}^{1 / 2}} .$$

The input referred voltage noise was calculated as

$$\frac{183 nV}{{Hz}^{1 / 2}}$$

which meets the 60 dB dynamic range requirement over the signal bandwidth of 2.25 kHz.

### Signal Processing and Feature Classification Strategies for Acoustic Detection of Vascular Stenosis

As provided herein, phonoangiograms can be efficiently transduced through arrays of flexible microphones, and can provide the bandwidth and dynamic range needed for interface and data conversion electronics. After a bruit is recorded, a wide range of digital signal processing strategies can be used to extract meaningful features. Prior examples have reported that autoregressive spectral envelope estimation, wavelet sub-band power ratios, and wavelet-derived acoustic features correlate to degree of stenosis. Features can be extracted from multiple signal processing branches and compared using machine-learning techniques, e.g. radial basis functions or random forests. However, feature extraction and model training must be constrained to prevent over-fitting on limited datasets, and to improve generalized use. As described herein, two derived time-domain signals—acoustic spectral centroid (ASC) and acoustic spectral flux (ASF)—have unique properties for bruit classification. Importantly, ASC and ASF can be derived directly from the discrete wavelet transform coefficients, which reduces feature dimensionality and aids scalar feature extraction.

A specific physical system implementation provides constraints on computational complexity, accuracy, and ease of implementation which can guide the selection of features. Described herein is a fundamental approach for extracting spectral features from a single acoustic recording site. As further described herein, signal processing can be further expanded into other domains, specifically into time and space, by leveraging time-synchronized recordings from an array of microphones.

### Multi-Domain Phonoangiogram Feature Calculation

Because PAGs are time-domain waveforms, they can be analyzed in both the temporal or spectral domains, i.e. as one-dimensional signals in either domain. Spectral transforms such as discrete cosine transform and continuous wavelet transform combine these domains to form a two-dimensional waveform along time and frequency (or scale) axes. However, when PAGs are acquired at multiple sites along a vascular access, the spatial distribution of PAG properties provides an additional analysis domain. If PAGs are also sampled simultaneously, time-domain differences between signals can be correlated and can be analyzed. When features are extracted from different domains, they can be compared to each other using clustering and classifier techniques as long as they are reduced to scalar form.

This section explains how features can be extracted from each domain with dimensional reduction to scalar values. The spectral domain provides scalar features such as average pitch. The temporospectral (combined time-spectral) domain allows segmentation of blood sounds in cardiac cycles to provide sample indices for systole onset. After temporospectral segmentation, spectral features can be separately calculated in systolic and diastolic phases. Finally, the spatial domain provides features describing the time delay between PAGs at different recording sites. Spatial analysis also enables detection of spectral changes between sites to predict where turbulent blood flow is occurring.

### Spectral-Domain Feature Extraction

Spectral-domain feature extraction is likely the most common approach in PAG signal processing. This is intuitive because humans perceive frequency content with great sensitivity, and PAG processing seeks to replicate traditional auscultation by ear. This section includes a review of spectral-domain feature extraction using continuous wavelet transform (CWT) to describe the spectral variance over time.

CWT over k scales W[k, n] is computed as

$$W [ k , n ] = {x}_{PAG} [ n ] * ψ [ n / k ] ,$$

where ψ[t/k] is the analyzing wavelet at scale k and x_{PAG} [n] is the PAG for each sample n. A complex Morlet wavelet was used because it has good mapping from scale to frequency, defined as

$$ψ [ n ] = {e}^{- {( n / 2 {f}_{c} )}^{2}} {e}^{j 2 π {f}_{c} n} ,$$

where f_{c} is the wavelet center frequency. In the limit f_{c}→∞, the CWT with Morlet wavelet becomes a Fourier transform. Because of the construction of the Morlet wavelet as the wavelet ψ[n] is scaled to ψ[n/k], and k is a factor of 2, the wavelet center frequency will be shifted by one octave. Therefore, CWT analysis with the Morlet wavelet can be described by the number of octaves (N_{O}) being analyzed (frequency span) and the number of voices per octave N_{V} (divisions within each octave, i.e. frequency scales). Mathematically the set of scale factors k can be expressed as

$$\begin{matrix}k [ {i}_{O} , {i}_{V} ] = {2}^{( {i}_{O} + {i}_{V} / {N}_{V} )} . \\ {i}_{O} = {k}_{0} , {k}_{0} + 1 , {k}_{0} + 2 … {N}_{O} \\ {i}_{V} = {k}_{0} , {k}_{0} + 1 , {k}_{0} + 2 … {N}_{V}\end{matrix}$$

Where k₀ is the starting scale and defines the smallest scale value and the total number of scales K=N_{O}N_{V}. For PAG analysis, CWT was computed with N_{O}=6 octaves and N_{V}=12 voices/octave, starting at k₀=3. After computing the CWT, pseudofrequencies F[k] across all K scales are calculated as

$$F [ k ] = {f}_{c} / k .$$

Because the CWT involves time-domain convolution, each discrete sample n has a paired sequence of k CWT coefficients, i.e. it is a 2-dimensional sequence. In the context of phonoangiogram classification, features can be extracted from W[k, n] that are of singular dimension. Dimension reduction of W[k, n] can operate over all or part of the k scales at each discrete sample n, over a single k scale for all n samples, over all points of W[k, n], or through a more complex combination of summation over k and n.

The systolic and diastolic portions of pulsatile blood flow contain differing spectral information on turbulent flow, so the CWT dimensionality can be reduced to n to produce time-domain waveforms. This preserves the spectral differences between different times in the cardiac flow cycle. Two n-point waveforms are calculated from W[k, n]: Auditory Spectral Flux (ASF) and Auditory Spectral Centroid (ASC). From these waveforms, time-independent features such as RMS spectral centroid can be computed, or time-domain spectral features can be extracted as explained in the next section.

ASF describes the rate at which the magnitude of the auditory spectrum changes, and approximates a spectral first-order derivative. It is calculated as the spectral variation between two adjacent samples, i.e.

$$ASF [ n ] = \frac{1}{K} \sqrt{{\sum }_{k = 1}^{K} {( \vert W [ k , n ] \vert - \vert W [ k , n - 1 ] \vert )}^{2}}$$

where W[k, n] is the continuous wavelet transform obtained over k total scales.

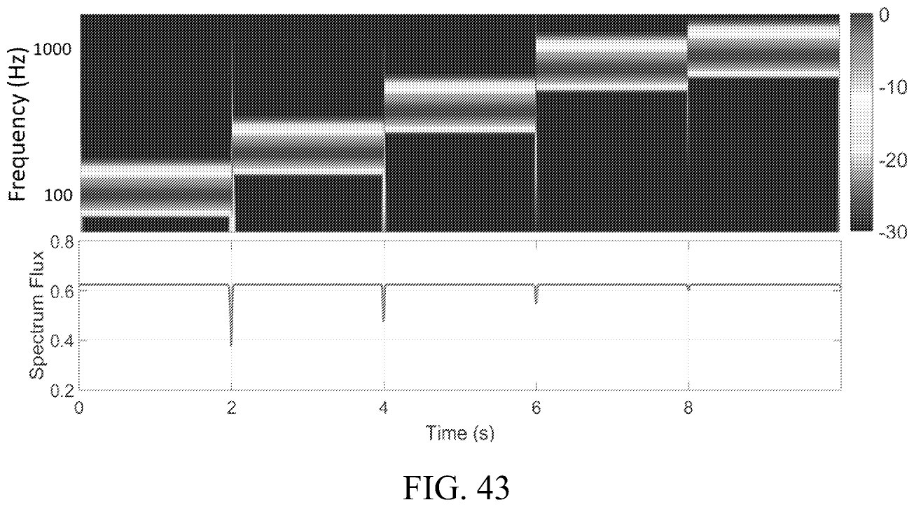

To intuitively demonstrate how ASF describes a signal, FIG. 43 shows ASF calculated from a stepped single tone test waveform. The tone changes over [100 200 400 800 1000] stepping every 2 seconds. At every tonal change, the spike in the ASF waveform corresponds to the time of the spectral shift, and the magnitude. The ASF waveform, therefore, describes how when, and how quickly, spectral power is shifting between bands. This is useful in mapping large variations in a PAG signal, such as the systole and diastole phases. Segmentation of these phases, therefore, uses the ASF waveform (described below).

ASC describes the spectral “center of mass” at each n sample in time. For Gaussian-distributed white noise, ASC will be constant at pseudofrequency F[K/2]. ASC is commonly used to estimate the average pitch of audio recordings, where a higher value corresponds to “brighter” acoustics with more high frequency content. ASC is calculated as:

$$ASC [ n ] = \frac{{\sum }_{k = 1}^{K} ( \vert W [ k , n ] \vert \cdot fc [ k ] )}{{\sum }_{k = 1}^{K} \vert W [ k , n ] \vert}$$

Where W[k, n] is the continuous wavelet transform obtained over K total scales of the PAG and f_{C}[k] is the center frequency.

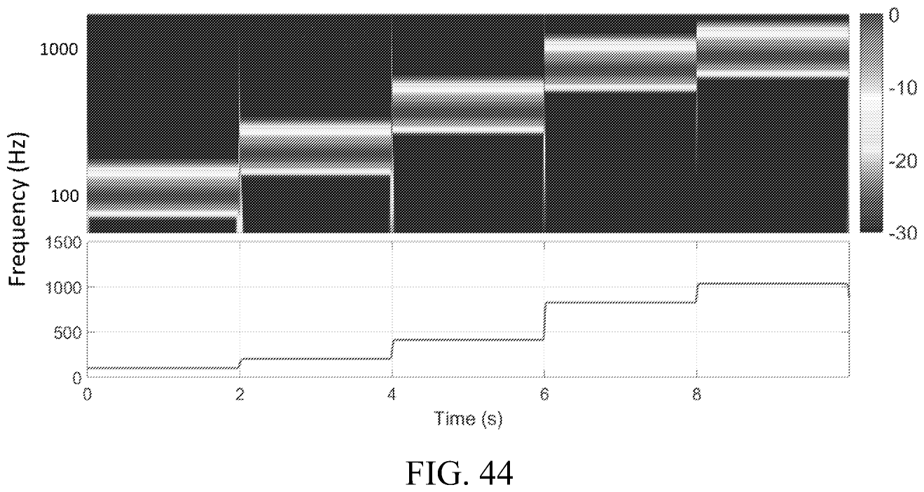

ASC for the same test waveform is plotted to intuitively describe how this waveform describes the time-domain spectral energy of a signal (FIG. 44). Because only a single tone is used at each time point, ASC consistently describes the frequency of the sine wave until it changes. Because F[k] represents pseudofrequencies, there is not a perfect mapping between ASC pseudofrequency and real auditory frequency. The use of the Morlet waveform in the CWT improves the pseudofrequency accuracy, but for PAG classification absolute frequency accuracy is not needed (discussed below).

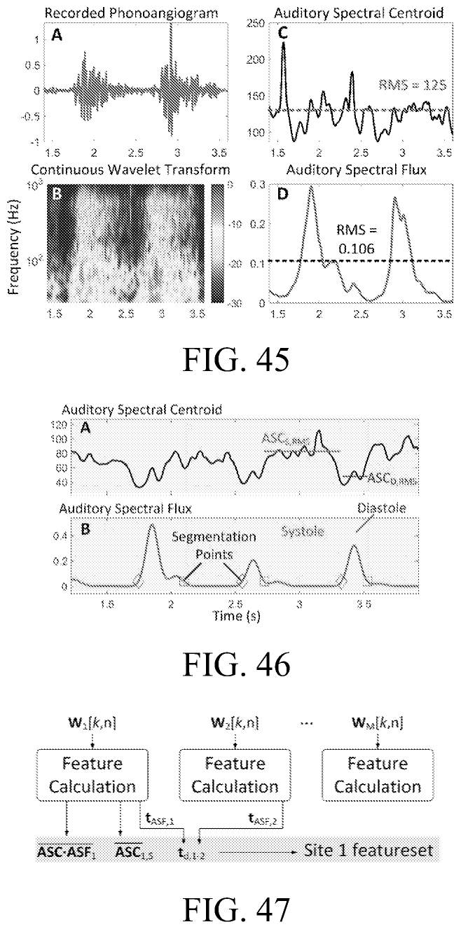

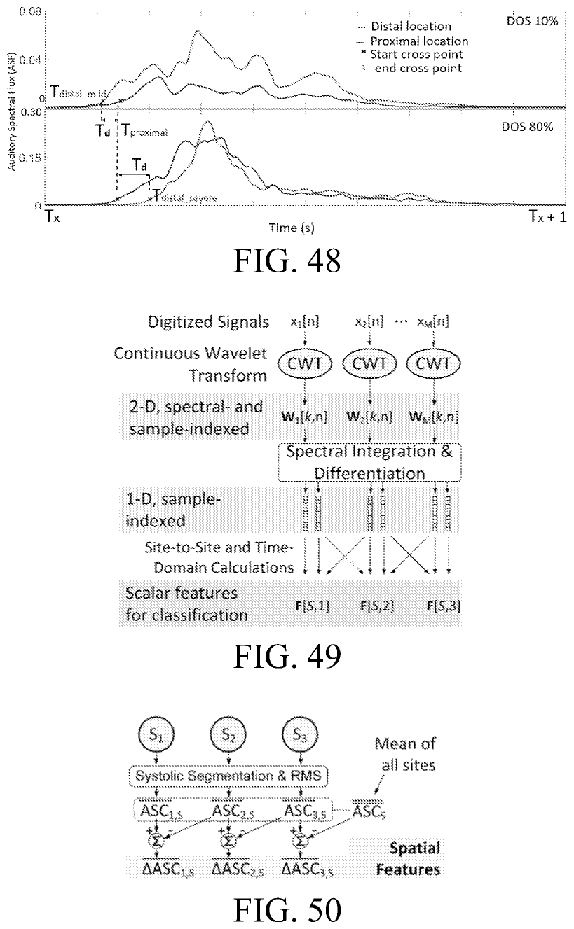

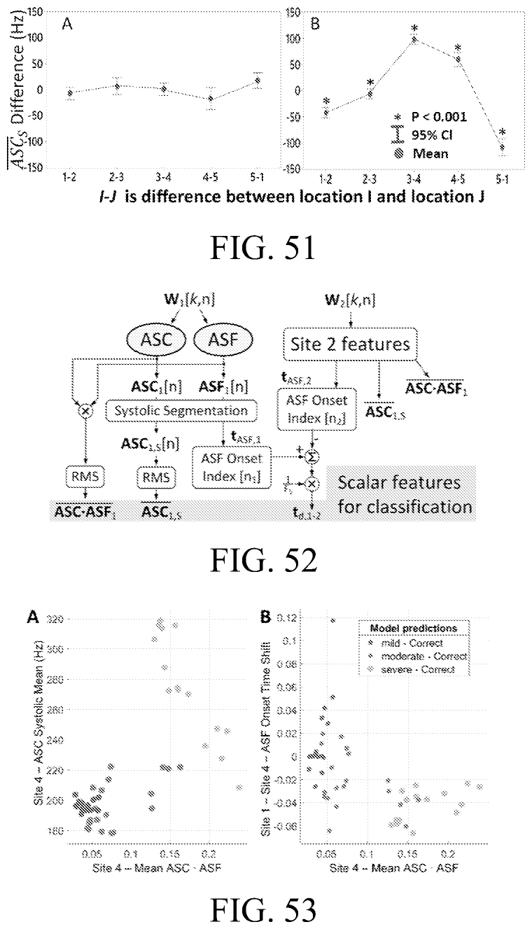

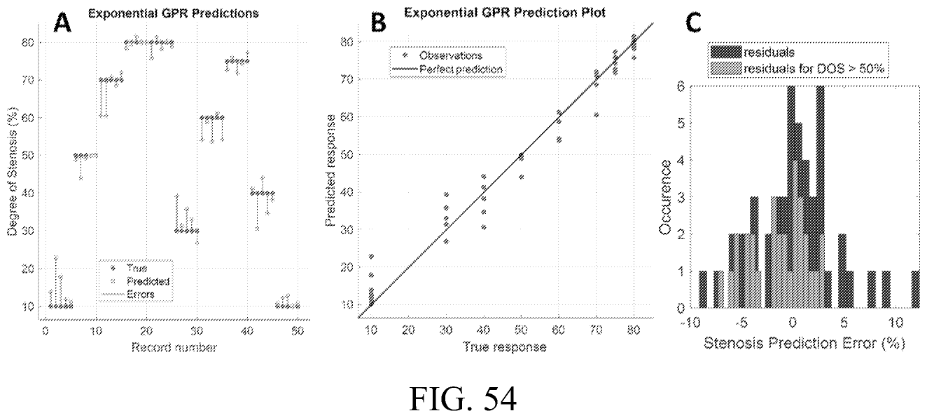

Example computations of ASC and ASF waveforms, compared to the time-domain and spectral-domain PAG recording demonstrate feature calculation (FIG. 45). After the 3-dimensional W[k, n] is computed, time-domain ASC and ASF waveforms are calculated. From these waveforms simple, time-invariant scalar values such as RMS or peak amplitude are calculated and used for stenosis classification.

### Temporospectral-Domain Feature Extraction

For PAG analysis, the primary interest is identifying the time onset of systolic and diastolic phases. This allows separate spectral feature extraction in each phase, ratioed features by comparing spectral changes between phases, and time-domain comparisons such as lengths of cardiac phases, or time shifts between recording sites. This analysis is useful because blood flow acceleration occurs in the high-pressure systolic pulse, which gives rise to turbulence producing high spectral power. As a spectral derivative, the ASF waveform is well suited to describe the onset of systolic turbulence and is used for temporospectral segmentation.

Segmentation can simply use a thresholding procedure; systolic ASF onset can be defined as the time when the ASF waveform exceeds a threshold in each pulse cycle (FIG. 46). A suitable threshold of 25% of the ASF_{RMS} value was determined empirically using data recorded from human patients and the vascular phantom. Pulse width is also used to reduce false threshold crossings. The times between threshold crossings can be calculated and any crossings which produce pulse widths less than 40% of the mean are discarded.

Temporospectral segmentation can produce a set of i indices (n_{ASF,i}) describing systolic and diastolic pulse widths, which themselves can be used as features. However, the indices can also be used to segment spectral waveforms such as ASF and ASC to split them into systolic ASFs and ASC_{S}, and diastolic ASF_{D} and ASC_{D}. Features for each phase can be calculated by combining all segments, or by averaging the feature for each segment. As an example, consider an ASC waveform segmented into P systolic segments each with length n. The RMS value of ASC in the systolic phase only is then

$${ASC}_{S , RM S} = \frac{1}{P} {\sum }_{i = 1}^{P} \sqrt{\frac{1}{n} \sum {ASC}_{S , P , n}^{2}}$$

In practice, because systolic segments do not all have the same length n, any derived features are calculated for each segment independently and averaged over P segments.

Ratiometric features can also be calculated as ratios or differences between successive systolic/diastolic pairs. This reduces the effect of interference caused by recording which is correlated between adjacent segments, or can be a less individual-specific feature because the diameter of the blood vessel and absolute flow rate contribute to ASC and differ between people. For example, ASC and ASF waveforms show significant differences in systolic and diastolic phases (FIG. 46), especially as DOS increases.

### Spatial-Domain Feature Extraction

The final domain analyzed in this model of PAG signal processing can be the spatial domain. Features are not extracted directly from the spatial domain, rather, new features are derived as the difference in features between sites (FIG. 47). This is a powerful technique because not only does it accentuate regions of turbulent flow, the proportional feature changes between recording locations are themselves related to degree of stenosis. Therefore, spatial-domain features are useful for both physical localization of stenosis, and classification of degree of stenosis. Furthermore, ratiometric site-to-site feature comparisons remove some of the individual variation in features attributed to differences in anatomy. For example, the dimensionless change in systolic ASC (ASC_{S}) between sites 1 and 2 can be calculated as

$${ASC}_{2 , S} / {ASC}_{1 , S} .$$

To obtain a similar comparison in approximate units of Hertz, a difference is used, i.e. ASC_{2,S}−ASC_{1,S}.

This spatial-domain technique can be generalized to produce composite features for any multi-site measurement with little complications as long as the compared features are independent scalars. However, any site-to-site calculations relying on time require synchronization in sample rates between sites, or alignment of waveforms based on a reference symbol so that relative time differences can be calculated. For example, composite temporospectral features require time invariance in the calculation. Once this condition is met, composite spatial-domain features based on time shifts are simple to calculate. For example, the time delay in ASF systolic onset (n_{ASF}) between sites 1 and 2 can be calculated as

$${t}_{d , 1 - 2} = 1 / {F}_{s} ( {n}_{ASF , 1} - {n}_{ASF , 2} ) .$$

This calculation is easily performed from feature calculations for each recording site (FIG. 38) and is transformed to a continuous time difference in units of seconds by dividing by the sample rate F_{S}. Scalar features from multiple domains can be combined to form a single featureset (FIG. 37), especially if a machine-learning classifier will be used because the scalar features can be analyzed as if they are unitless.

### Classification of Vascular Access Stenosis Location and Severity In Vitro

A clinical goal for multi-site recordings of PAGs can be to both locate and describe the severity of stenosis. It has been shown that binary or ternary classification using single features can be sufficient to classify DOS as mild, moderate, or severe. Analysis of this method using receiver operating characteristic (ROC) revealed detection sensitivities as high as 88-92% and specificities as high as 96-100%, but classification was only accurate at certain recording locations. Therefore, feature selection for an array of recording sites is important to detect differences between recording sites. This section demonstrates comparing features between sites using hyperdimensional classifiers to greatly improve the stenosis classification accuracy from PAG recordings.

### Multi-Domain Feature Selection

The previous sections described how phonoangiograms are transduced and processed as analog signals, prior to being digitized for digital signal processing. Features are then extracted from multiple dimensions to yield a final set of M features F[S.M], which are site-specific to each of S recording sites (FIG. 39). Previous work has described more than 15 features that are correlated with degree of stenosis in humans and in bench phantoms of vascular stenosis.

Machine-learning classifiers can use optimized feature selection through numerous methods. Feature selection can improve the performance of classifier algorithms and reduce the likelihood of over-fitting to a data set of limited size. Numerical methods such as principal component analysis are powerful tools, as is supervised feature selection which relies on trained experts to select the features describing most of the variance in the observed effect. This work used both automated and supervised feature selection to select the most appropriate features. The following classification examples explain the rationale behind feature selection for the given classification task.

### Stenosis Spatial Localization Using Acoustic Features

Because the presence of stenosis produces turbulent flow in blood, a characteristic high frequency sound can be produced locally within 1-2 cm of the lesion. Spatial-domain feature analysis can be advantageous to detect differences between recording sites caused by dramatic changes in blood flow patterns. To demonstrate the feasibility of detecting the location of stenosis using acoustic features alone, 8 stenosis phantoms on the vascular phantom previously described were tested over variable blood flow rates of 700-1,200 mL/min. This range of flows was tested at each degree of stenosis to simulate the nominal levels of human blood flow rates in arteriovenous vascular accesses. DOS for the phantoms ranged from 10-85%.

A vascular access is typically a uniform segment of blood vessel with few collateral veins. Accordingly, a one-dimensional recording array with 5 locations along the path of blood flow (FIG. 4) was used for testing. Recording sites were spaced by 1 cm and used skin-coupled microphones as previously described. While over 15 features were analyzed for stenosis localization, many features were correlated, and therefore adopted the site-to-site change in mean systolic ASC (Δ̅A̅S̅C̅_̅{̅S̅}̅) as the sole feature for localization (FIG. 19). This feature was intuitively selected because it is well documented that the presence of stenosis causes high-pitched blood sounds. Therefore, it was expected that an abrupt stenosis in an otherwise smooth vessel produces higher pitch at sites within several centimeters. Five site-to-site features for each flow rate and DOS were calculated, and including replications this yielded 370 total samples for statistical analysis.

In this experiment, the actual stenosis was located directly under location 2; location 1 was recorded 1 cm proximal, and locations 3, 4 and 5 were 1, 2 and 3 cm distal to stenosis. The interval plot (FIG. 20) indicates a positive shift between Δ̅A̅S̅C̅_̅{̅S̅}̅, differences from proximal to distal locations (p<0.001 for 30%<DOS<90%). Confidence intervals and differences in group means were calculated using ANOVA followed by Tukey's test with 95% confidence intervals (u=0.05). Because sample data followed a normal distribution, Tukey's test was used to adjust confidence intervals based on the number of comparisons tested. Statistical analysis was performed in Minitab software (Minitab, LLC, State College, PA, USA). In general, differences between locations 3 and 4 and 4 and 5 were positive by 50-70 Hz while the other site differences were negative. This indicates that a simple threshold difference of 70 Hz in Δ̅A̅S̅C̅_̅{̅S̅}̅, between adjacent array recording locations can identify stenosis proximally to the recording sites within 1-2 cm.

Stenosis Severity Classification from Acoustic Features

While the location of stenosis can be estimated by comparing feature shifts between sites to a threshold, classification of the degree of stenosis can be more challenging from a single feature. This is in part because the degree of stenosis and the nonlinear properties of blood interact such that DOS nonlinearly impacts overall flow rate and turbulence pattern, introducing time-dependent changes to both acoustic spectra and intensity. Many classification strategies have been proposed and studied for a single recording site (e.g., showing classification accuracy of about 84% using binomial Gaussian modeling). It is further contemplated that classification can be extended to leverage temporo-spatial domain features drawn from multiple recording sites.

PAG data can be classified using a quadratic support vector machine (SVM). The quadratic SVM is widely used in natural language processing tasks, and is suitable for PAGs which have similar autoregressive properties as speech. As a machine-learning algorithm, the SVM can define a hyperplane that is used to separate clusters of data points in a high-dimensional space. The hyperplane can be used as a decision surface and can be optimized to maximize the separation distance between the classes of data.

Because the data are not linearly separable, the SVM can transform the input data points into a higher dimension using a kernel function. For the quadratic SVM the kernel K is a polynomial of order 2, i.e.

$$K ( {x}_{1} , {x}_{2} ) = {( {x}_{1}^{T} {x}_{2} + 1 )}^{2} .$$

Expanding this kernel reveals how data are expanded into higher dimension through interaction terms:

$$K ( {x}_{1} , {x}_{2} ) = {( {\sum }_{i = 1}^{n} {x}_{1}^{T} {x}_{2} + 1 )}^{2} = {\sum }_{i = 1}^{n} {x}_{1 , i}^{2} {x}_{2 , i}^{2} + {\sum }_{i = 1}^{n} {\sum }_{j = 1}^{i - 1} ( \sqrt{2} {x}_{1 , i} {x}_{1 , j} ) ( \sqrt{2} {x}_{2 , i} {x}_{2 , j} ) + {\sum }_{i = 1}^{n} ( \sqrt{2} {x}_{1 , i} ) ( \sqrt{2} {x}_{2 , i} ) + 1.$$

This dimensional expansion can change the distances between data points in the higher-dimensional space and allows a decision surface to be constructed. The decision surface can be a hyperplane optimized to the distance between the hyperplane and the nearest data points in each class. Because this quadratic optimization problem involves significant computation, SVM can be developed using machine-learning strategies and can be generally tuned iteratively.

For the case of DOS classification, the SVM can be trained in MATLAB (or other suitable software) using the same dataset of 370 recordings described above. For each of S recording sites a set of M features was calculated giving a total feature array F[S,M]. However, after detecting the location of stenosis, only recordings from the nearest site need to be classified. For example, the SVM can be only trained on a single feature vector F[M]. In our example with 5 recording sites, this reduced the total number of observations (recordings) to 50.

In certain examples, training of the SVM was performed in MATLAB in three phases. First, PAG features were transformed to a high-dimensional space using the polynomial kernel. Then, feature selection was performed to reduce the total number of features (and hence the dimensionality) of the SVM. This reduced the overall model complexity, reduced the numerical instability risk inherent to SVMs, and reduced the risk of over-fitting. Principal component analysis can be used to define the three features which described variance between the data classes: A̅S̅C̅·̅A̅S̅F̅ (Mean ASC multiplied by Mean ASF), A̅S̅C̅_̅{̅S̅}̅ (Mean value of ASC in systole), and t_{d} (time shift in ASF onset compared to first recording site). The computation of these features is illustrated in FIG. 52. Then, quadratic optimization was performed to fit an optimal hyperplane between the classes of data. Model validation was performed using five-fold cross validation such that the model was trained on 10 observations and tested by classifying the remaining 40.

The quadratic SVM can be designed to classify PAGs into three output classes for DOS: mild, moderate, and severe. Because, in exemplary testing, these classes were ordinal (monotonic) and known a priori, quadratic SVM were selected (versus, for example, clustering methods). Further, while DOS is a continuous variable, DOS was organized or binned into classification ranges because clinical monitoring does not require precise quantification of DOS; imaging is then used after a lesion is identified to more precisely determine treatment options. However, acoustic features can also be used to continuously estimate the DOS using regression, as described in the following section. Thresholding after regression can be used to similarly classify estimated DOS into ranges for clinical action.

Class definitions were chosen (e.g., DOS<30% (mild), 30%≤DOS≤70% (moderate), and DOS>70% (severe)). Validation accuracy of the quadrative SVM on this data was 100% even though the features were not linearly separable (FIG. 53). Importantly, as reflected in Table 3, most of the classification accuracy came from the ASC and ASF features, however, adding the temporo-spatial measure t_{d} helped prevent misclassifications at high DOS which occur when the stenosis greatly reduces vascular flow rate.

TABLE 3
Performance of Quadratic SVM versus included features

| Number of | | Average Validation |
|---|---|---|
| Features | Included Features | Accuracy |
| 3 | A̅S̅C̅ ·̅ A̅S̅F̅, | 100% |
| | A̅S̅C̅_̅{̅S̅}̅, t_{d} | |
| 2 | A̅S̅C̅ ·̅ A̅S̅F̅, | 96% |
| | A̅S̅C̅_̅{̅S̅}̅ | |
| | A̅S̅C̅_̅{̅S̅}̅, t_{d} | 84% |
| | A̅S̅C̅ ·̅ A̅S̅F̅, t_{d} | 88% |
| 1 | A̅S̅C̅ ·̅ A̅S̅F̅ | 84% |
| | A̅S̅C̅_̅{̅S̅}̅ | 82% |
| | t_{d} | 48% |

However, while t_{d} boosts classification accuracy only slightly, multiple recording locations for stenosis localization is still essential to accurate classification. For example, Table 4 indicates how classification accuracy drops significantly when applied to PAGs recorded more than 2 cm from the actual site of stenosis and dropping the spatial feature t_{d}. This indicates that accurate PAG classification requires either a priori knowledge of stenosis location or multi-site recordings to detect locations for analysis.

TABLE 4
Classifier Accuracy of Quadratic SVM versus single recording sites

| Recording Site | A̅S̅C̅ ·̅ A̅S̅F̅ | A̅S̅C̅_̅{̅S̅}̅ | A̅S̅C̅ ·̅ A̅S̅F̅, A̅S̅C̅_̅{̅S̅}̅ |
|---|---|---|---|
| 1 | 70% | 68% | 66% |
| 2 | 70% | 68% | 78% |
| 3 | 60% | 54% | 86% |
| 4 | 84% | 84% | 96% |

This analysis suggested that machine-learning can be used for accurate classification of PAGs. The model was trained using data from a set of vascular phantoms with variable rates of blood flow, but it is understood that additional data and/or classification may be necessary to account for the wide anatomical variance seen in humans.

Degree of Stenosis Estimation from Acoustic Features

In addition to using acoustic features from PAGs to classify stenosis into clinically actionable ranges, acoustic features can also be used to predict the actual degree of stenosis. It is contemplate that DOS could be estimated within 6% given a priori knowledge of the stenosis location. As shown herein, features from multiple domains can be used to further improve DOS estimation using Gaussian process regression (GPR).

GPR is a regression modeling method, but unlike linear or nonlinear regression—which seeks to fit a least-squares model to minimize prediction error to a dataset f(x)—GPR is a Bayesian process which models f(x) as a Gaussian process. Thus, the value f(x) at each point x is represented as a random variable with a Gaussian distribution. The actual values used to train the model are therefore considered simply as independent observations drawn from the underlying normal probability distribution at each point. For example, observation-response pairs (x₁,y₁) and (x₂,y₂) are represented by normal distributions P(y₁|x₁) and P(y₂|x₂). Regression of a new response y₃ based on a new observation x₃ is then calculated as the conditional probability P(y₃|(y₁, y₂), (x₁, x₂, x₃))

Assuming the mean of the joint distribution of all input features F[M] is zero (accomplished through normalization without losing information between each recording), training the GPR involves solving for the unknown covariance matrix using a radial basis function kernel K(xₘ, xₙ), i.e.

$$Cov ( f ( {x}_{m} ) , f ( {x}_{n} ) ) = K ( {x}_{m} , {x}_{n} ) = {α}^{2} {e}^{- \frac{1}{2 {l}^{2}} {( {x}_{m} - {x}_{n} )}^{2}} .$$

In this example, the parameter α² is the output variance of the data while l² represents the lengthscale of the data variance. Generally, α² indicates the average distance of the function from its mean, while l determines the memory length of the modeled GPR. For a GPR trained on time-invariant features, e.g. PAG features, l=1. Similarly to the quadratic SVM, training data are transformed by the basis function to a higher-dimensional space. Optimization of the basis function is then performed iteratively to minimize the RMS predicted error to the input data. Model training was performed in MATLAB on the same 50 recordings used to train the quadratic SVM classifier. As reflected in Table 5, the RMS error of the optimized GPR was calculated using five-fold cross-validation.

TABLE 5
RMS error from exponential GPR model versus
features and recording sites included

| | Features included in model | | | |
|---|---|---|---|---|
| Sites included | | | A̅S̅C̅ ·̅ A̅S̅F̅, | A̅S̅C̅ ·̅ A̅S̅F̅, |
| in model | A̅S̅C̅ ·̅ A̅S̅F̅ | A̅S̅C̅_̅{̅S̅}̅ | A̅S̅C̅_̅{̅S̅}̅ | A̅S̅C̅_̅{̅S̅}̅, t_{d} |
| Site 1 only | 20.0% | 16.6% | 10.1% | 9.4% |
| Site 2 only | 20.0% | 19.0% | 12.9% | 9.2% |
| Site 3 only | 21.3% | 22.0% | 8.6% | 7.5% |
| Site 4 only | 13.8% | 16.9% | 12.2% | 8.0% |
| All sites | 8.9% | 7.9% | 5.2% | 4.3% |
| included | | | | |

While the SVM classifier was demonstrated in the previous section, SVM regression was not used for stenosis estimation. GPR was selected after feature distribution analysis, which indicated that due to the chaotic nature of turbulent fluid flow, and the dependency on variable blood flow rate, features measured at each degree of stenosis spanned a range of observations around a defined central value. Generally, for DOS>50% extracted features followed a normal distribution when pooled across all recording sites and all flow rates. Although GPR would suffer from finite bounds on confidence intervals because DOS is bounded on the range of 0-100%, because the model was only validated on the range of DOS from 10-90%, GPR out-performed other regressions, perhaps due to estimation of the underlying variance for each feature. For example, using the same features as in Table 5, quadratic SVM regression only achieved a best-case 8.3% RMS error.

As in the quadratic SVM classifier, the addition of more features reduced the RMS error of the regression. However, unlike the SVM, the regression required data from sites around the stenosis to improve accuracy. In this example, the actual stenosis lesion was located under Site 2 with turbulent flow occurring beneath Site 3 and Site 4 based on established models. Including features from recordings proximal and distal to the lesion greatly improved the estimation accuracy. For all tested DOS, error was in the range [−11% 14%], and for DOS>50% error was [−11% 3%](FIG. 44). This outcome led to two conclusions. First, this in vitro experiment clearly demonstrates the need for multiple recording sites for accurate phonoangiographic estimation of degree of stenosis. In humans with more variable vascular anatomy the need for the multiple recording sites is likely greater because the location or presence of stenosis is not known a priori. Second, the achieved accuracy is sufficient for clinical monitoring, which generally only needs to detect when stenosis exceeds 50% or is rapidly progressing.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: An apparatus for detecting acoustic signals of a vascular system, the apparatus comprising: at least one acoustic sensor comprising: a structure defining a hole therethrough; a piezoelectric polymer layer defining a first side and a second side, wherein the piezoelectric polymer layer extends across the hole of the structure; a first electrode disposed on the first side of the polymer layer; a second electrode disposed on the second side of the polymer layer; and a polymer engagement layer positioned against the first side of the polymer layer and disposed at least partially within the hole of the structure.

Aspect 2: The apparatus of aspect 1, wherein the structure defining the hole therethrough comprises the first electrode, wherein the first electrode is annular and defines a first opening that is coaxial with the hole, and wherein the second electrode is annular.

Aspect 3: The apparatus of aspect 2, wherein the second electrode defines a second opening that is coaxial with the first opening of the first electrode.

Aspect 4: The apparatus of any one of the preceding aspects, wherein the piezoelectric layer extends across the first opening of the first electrode and comprises PVDF.

Aspect 5: The apparatus of aspect 4, wherein the piezoelectric layer comprises silver ink-metallized PVDF film.

Aspect 6: The apparatus of claim 1, wherein the polymer engagement layer comprises PDMS.

Aspect 7: The apparatus of claim 1, wherein the hole of the structure has a diameter of between 1 and 3 mm.

Aspect 8: The apparatus of claim 1, wherein the hole of the structure has a diameter of about 2 mm.

Aspect 9: The apparatus of claim 2, wherein the apparatus comprises a first polyimide printed circuit board and a second polyimide printed circuit board, wherein the first electrode of each of the at least one acoustic sensor is a component of the first polyimide printed circuit board and the second electrode of each of the at least one acoustic sensor is a component of the second polyimide printed circuit board, and wherein the structure that defines the hole comprises the first polyimide printed circuit board.

Aspect 10: The apparatus of claim 1, further comprising an outer layer disposed on the second side of the piezoelectric layer.

Aspect 11: The apparatus of claim 9, wherein the outer layer comprises silicone gel.

Aspect 12: The apparatus of claim 1, wherein the at least one acoustic sensor comprises a plurality of acoustic sensors disposed in a spaced relationship along a first axis.

Aspect 13: The apparatus of claim 12, wherein the plurality of acoustic sensors are spaced center-to-center from sequential acoustic sensors along the first axis by about one centimeter.

Aspect 14: The apparatus of claim 12, wherein the structure defines gaps between outer edges of sequential acoustic sensors of the plurality of acoustic sensors to reduce cross-talk between the sequential acoustic sensors.

Aspect 15: The apparatus of claim 1, further comprising a front end that is configured to receive an analog signal from the at least one acoustic sensor and process the analog signal to provide a modified signal.

Aspect 16: The apparatus of claim 15, wherein the front end comprises a trans-impedance amplifier that is configured to convert a current to a voltage and a low-pass filter.

Aspect 17: The apparatus of claim 16, wherein the front end comprises a multiple feedback filter comprising the low-pass filter, wherein the low-pass filter is configured to limit pole splitting.

Aspect 18: A method comprising: applying a bruit enhancing filter to data collected using an apparatus as in any one of aspects 1-17 to generate bruit enhanced filtered data; and applying a wavelet transform to the bruit enhanced filtered data to provide wavelet data.

Aspect 19: The method of aspect 18, further comprising: generating an auditory spectral flux waveform (ASF) from the wavelet data; and generating an auditory spectral centroid waveform (ASC) from the wavelet data.

Aspect 20: The method of aspect 19, further comprising: performing a systole/diastole segmentation on the auditory spectral flux waveform and the auditory spectral centroid waveform.

Aspect 21: The method of aspect 20, wherein performing the systole/diastole segmentation on the auditory spectral flux waveform and the auditory spectral centroid waveform comprises calculating at least one of: a mean value of a systole segment of the ASC, a root mean square (RMS) of a systole segment of the ASF, a difference between the mean value of the systole segment of the ASC and a mean value of a diastole segment of the ASC, or a product of the mean of the systole segment of the ASC and the RMS of the systole segment of the ASF.

Aspect 22: The method of aspect 21, further comprising: determining a first time of a crossing of a threshold of the ASF for data from a first sensor; determining a second time of a crossing of the threshold of the ASF for data from a second sensor that is distal to the first sensor with respect to a blood flow direction; and calculating a difference between the first time and the second time.

Aspect 23: The method of aspect 22, further comprising determining a degree of stenosis based on the difference between the first time and the second time.

Aspect 24: The method of claim 20, further comprising performing a regression on the ASC, the ASF, and time data to determine a degree of stenosis (DOS).

Aspect 25: The method of claim 24, wherein the regression is a Gaussian process regression.

Aspect 26: The method of claim 24 or claim 25, further comprising using machine learning classifiers to classify the DOS within at least one range.

Aspect 27: The method of claim 26, wherein the at least one range comprises mild, moderate, and severe.

Aspect 28: The method of claim 26 or claim 27, wherein the machine learning classifiers comprise a support vector machine.

Aspect 29: A system comprising: an apparatus as in any one of aspects 1-17; and a computing device, wherein the computing device comprises at least one processor and a memory in communication with the at least one processor, wherein the memory comprises instructions that, when executed by the at least one processor, perform the method of any one of aspects 18-28.

Although the foregoing invention has been described in some detail by way of illustration and example for purposes of clarity of understanding, certain changes and modifications may be practiced within the scope of the appended claims.

1. An apparatus for detecting acoustic signals of a vascular system, the apparatus comprising:
at least one acoustic sensor comprising:
a structure defining a hole therethrough;
a piezoelectric layer defining a first side and an opposing second side, wherein the piezoelectric layer extends across the hole of the structure;
a first electrode disposed on the first side of the piezoelectric layer;
a second electrode disposed on the second side of the piezoelectric layer; and
a polymer engagement layer positioned against the first side of the piezoelectric layer and disposed at least partially within the hole of the structure.

2. The apparatus of claim 1, wherein the structure defining the hole therethrough comprises the first electrode, wherein the first electrode is annular and defines a first opening that is coaxial with the hole, and wherein the second electrode is annular.

3. The apparatus of claim 2, wherein the second electrode defines a second opening that is coaxial with the first opening of the first electrode.

4. The apparatus of claim 1, wherein the piezoelectric layer extends across the first opening of the first electrode and comprises PVDF.

5. (canceled)

6. (canceled)

7. (canceled)

8. (canceled)

9. The apparatus of claim 2, wherein the apparatus comprises a first polyimide printed circuit board and a second polyimide printed circuit board, wherein the first electrode of each of the at least one acoustic sensor is a component of the first polyimide printed circuit board and the second electrode of each of the at least one acoustic sensor is a component of the second polyimide printed circuit board, and wherein the structure that defines the hole comprises the first polyimide printed circuit board.

10. (canceled)

11. (canceled)

12. The apparatus of claim 1, wherein the at least one acoustic sensor comprises a plurality of acoustic sensors disposed in a spaced relationship along a first axis.

13. (canceled)

14. The apparatus of claim 12, wherein the structure defines gaps between outer edges of sequential acoustic sensors of the plurality of acoustic sensors to reduce cross-talk between the sequential acoustic sensors.

15. The apparatus of claim 1, further comprising a front end that is configured to receive an analog signal from the at least one acoustic sensor and process the analog signal to provide a modified signal.

16. The apparatus of claim 15, wherein the front end comprises a trans-impedance amplifier that is configured to convert a current to a voltage and a low-pass filter.

17. The apparatus of claim 16, wherein the front end comprises a multiple feedback filter comprising the low-pass filter, wherein the low-pass filter is configured to limit pole splitting.

18. A method comprising:
applying a bruit enhancing filter to data collected using an apparatus to generate bruit enhanced filtered data, the apparatus comprising:
at least one acoustic sensor comprising:
a structure defining a hole therethrough:
a piezoelectric layer defining a first side and an opposing second side,
wherein the piezoelectric layer extends across the hole of the structure;
a first electrode disposed on the first side of the piezoelectric layer;
a second electrode disposed on the second side of the piezoelectric layer; and
a polymer engagement layer positioned against the first side of the piezoelectric laver and disposed at least partially within the hole of the structure; and
applying a wavelet transform to the bruit enhanced filtered data to provide wavelet data.

19. The method of claim 18, further comprising:
generating an auditory spectral flux waveform (ASF) from the wavelet data; and
generating an auditory spectral centroid waveform (ASC) from the wavelet data.

20. The method of claim 19, further comprising:
performing a systole/diastole segmentation on the auditory spectral flux waveform and the auditory spectral centroid waveform.

21. The method of claim 20, wherein performing the systole/diastole segmentation on the auditory spectral flux waveform and the auditory spectral centroid waveform comprises calculating at least one of:
a mean value of a systole segment of the ASC,
a root mean square (RMS) of a systole segment of the ASF,
a difference between the mean value of the systole segment of the ASC and a mean value of a diastole segment of the ASC, or
a product of the mean of the systole segment of the ASC and the RMS of the systole segment of the ASF.

22. The method of claim 20, further comprising:
determining a first time of a crossing of a threshold of the ASF for data from a first sensor;
determining a second time of a crossing of the threshold of the ASF for data from a second sensor that is distal to the first sensor with respect to a blood flow direction; and
calculating a difference between the first time and the second time.

23. The method of claim 22, further comprising determining a degree of stenosis based on the difference between the first time and the second time.

24. The method of claim 20, further comprising performing a regression on the ASC, the ASF, and time data to determine a degree of stenosis (DOS).

25. The method of claim 24, wherein the regression is a Gaussian process regression.

26. The method of claim 24, further comprising using machine learning classifiers to classify the DOS within at least one range.

27. The method of claim 26, wherein the at least one range comprises mild, moderate, and severe.

28. The method of claim 26, wherein the machine learning classifiers comprise a support vector machine.

29. A system comprising:
an apparatus comprising:
at least one acoustic sensor con rising:
a structure defining a hole therethrough:
a piezoelectric layer defining a first side and an opposing second side, wherein the piezoelectric layer extends across the hole of the structure;
a first electrode disposed on the first side of the piezoelectric laver:
a second electrode disposed on the second side of the piezoelectric layer; and
a polymer engagement layer positioned against the first side of the piezoelectric layer and disposed at least partially within the hole of the structure; and
a computing device, wherein the computing device comprises at least one processor and a memory in communication with the at least one processor, wherein the memory comprises instructions that, when executed by the at least one processor, cause the at least one processor to:
apply a bruit enhancing filter to data collected using the apparatus; and
apply a wavelet transform to the bruit enhanced filtered data to provide wavelet data.
